(19) United States
(12) Patent Application Publication
(10) Pub. No.: US 2014/0288149 A1
(43) Pub. Date: Sep. 25, 2014

(54) MIR-142 AND ANTAGONISTS THEREOF FOR TREATING DISEASE

(71) Applicant: Graham Lord, London (GB)

(72) Inventor: Graham Lord, London (GB)

(21) Appl. No.: 14/207,851

(22) Filed: Mar. 13, 2014

Related U.S. Application Data
(60) Provisional application No. 61/788,902, filed on Mar. 15, 2013.

(51) Int. Cl. C12N 15/113

(52) U.S. Cl. CPC C12N 15/113
USPC 514/44 A

(57) ABSTRACT

Methods of treating various conditions using miR-142, miR-142 mimics, and antagonists of miR-142 are provided.

### 1. BACKGROUND

Until recently, it had been accepted that T cell lineage commitment was fixed during polarisation of a naïve CD4 T cell and that the fate of each lineage was controlled by the expression of a unique transcription factor. The transcription factors T-bet (Tbx21), GATA-3, ROR-gT and FoxP3 have been shown to play key roles in T helper type 1 (Th1), Th2, Th17 and regulatory T cell (Treg) lineages respectively (Szabo et al., 2000; Zheng and Flavell, 1997; Zhang et al., 1997; Ivanov et al., 2006; Hon et al., 2003; Khattri et al., 2003; Fontenot et al., 2003). However, there is increasing evidence that combinatorial expression of multiple lineage-specific transcription factors is important in controlling T cell differentiation (O'Shea and Paul, 2010). We have recently demonstrated that T-bet and GATA-3 are co-expressed in Th1 cells, and by identifying their transcriptional targets on a genome-wide scale have revealed that they co-ordinately bind to the promoters of many genes (Jenner et al., 2009). In addition, recent work has uncovered a range of situations in which T cell lineage commitment can be subverted to allow previously committed cells to switch lineages (Zhou et al., 2009). These findings have led us and others to propose that co-ordinate binding of transcription factors may be one of the mechanisms controlling T cell lineage specification and stability.

In addition, a number of factors that regulate T cell homeostasis have been extensively characterized, including cytokine signalling and engagement of T cell receptor (TCR) with self-peptide/MHC complexes. Many of these have specific effects on individual subpopulations, with naïve and memory CD4+ and CD8+ cells differing in their response to such stimuli. Cell-intrinsic pathways include regulation of the cell cycle, cell metabolism and both pro- and anti-apoptotic signals. However, current understanding of the regulation of these processes at the molecular level is limited.

MicroRNAs (miRNAs) are a class of short, non-coding RNAs that exhibit partial sequence complementarity with the mRNA of multiple target genes, and are capable of regulating expression of these genes post-transcriptionally. Multiple miRNAs are expressed in CD4⁺ T cells and inhibition of global miRNA expression by deletion of the endonuclease Dicer results in a number of functional abnormalities, including default hyperproduction of interferon (IFN)-γ (Muljo et al., 2005). MicroRNAs also play important roles in cell fate specification and plasticity of other lineages (Cordes et al., 2009).

### 2. SUMMARY

In some embodiments, methods of treating autoimmune diseases are provided. In some embodiments, a method comprises increasing levels of miR-142 in a subject with an autoimmune disease and/or administering a miR-142 mimic to a subject with an autoimmune disease. In some embodiments, the method comprises administering to the subject a vector that encodes an shRNA, wherein the shRNA comprises a region that is identical to at least 8 contiguous nucleotides of miR-142. In some embodiments, a method comprises administering to a subject with an autoimmune disease a compound comprising an oligonucleotide, wherein the oligonucleotide comprises a first strand that consists of 8 to 200 nucleosides, and wherein the first strand comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142. In some embodiments, the autoimmune disease is selected from rheumatoid arthritis, multiple sclerosis, psoriasis, and inflammatory bowel disease.

In some embodiments, methods of attenuating rejection of transplanted organs are provided. In some embodiments, a method comprises increasing levels of miR-142 in a subject who has received an organ transplant and/or administering a miR-142 mimic to a subject who has received an organ transplant. In some embodiments, the method comprises administering to the subject a vector that encodes an shRNA, wherein the shRNA comprises a region that is identical to at least 8 contiguous nucleotides of miR-142. In some embodiments, a method comprises administering to a subject who has received an organ transplant a compound comprising an oligonucleotide, wherein the oligonucleotide consists of 8 to 200 nucleosides, and wherein the oligonucleotide comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142. In some embodiments, the transplanted organ is selected from kidney, liver, lung, bone marrow, and heart.

In some embodiments, methods of enhancing IL-7 receptor signalling in a cell are provided. In some embodiments, a method comprises increasing levels of miR-142 in the cell and/or contacting a cell with a miR-142 mimic. In some embodiments, the method comprises contacting a cell with a vector that encodes an shRNA, wherein the shRNA comprises a region that is identical to at least 8 contiguous nucleotides of miR-142. In some embodiments, a method comprises contacting a cell with a compound comprising an oligonucleotide, wherein the oligonucleotide comprises a first strand that consists of 8 to 200 nucleosides, and wherein the first strand comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142.

In some embodiments, methods of enhancing IL-7 receptor signaling in a subject with HIV are provided. In some embodiments, a method comprises increasing levels of miR-142 in a subject with HIV and/or administering a miR-142 mimic to a subject with HIV. In some embodiments, the method comprises administering to the subject a vector that encodes an shRNA, wherein the shRNA comprises a region that is identical to at least 8 contiguous nucleotides of miR-142. In some embodiments, a method comprises administering to the subject a compound comprising an oligonucleotide, wherein the oligonucleotide consists of 8 to 200 nucleosides, and wherein the oligonucleotide comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142.

In some embodiments, methods of increasing regulatory T cell production in a subject are provided. In some embodiments, a method comprises increasing levels of miR-142 in a subject and/or administering a miR-142 mimic to a subject. In some embodiments, the method comprises administering to the subject a vector that encodes an shRNA, wherein the shRNA comprises a region that is identical to at least 8 contiguous nucleotides of miR-142. In some embodiments, a method comprises administering to the subject a compound comprising an oligonucleotide, wherein the oligonucleotide comprises a first strand that consists of 8 to 200 nucleosides, and wherein the first strand comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142.

In some embodiments, methods of increasing IgM antibody production in a subject are provided. In some embodiments, a method comprises increasing levels of miR-142 in a subject and/or administering a miR-142 mimic to a subject. In some embodiments, the method comprises administering to the subject a vector that encodes an shRNA, wherein the shRNA comprises a region that is identical to at least 8 contiguous nucleotides of miR-142. In some embodiments, a method comprises administering to the subject a compound comprising an oligonucleotide, wherein the oligonucleotide comprises a first strand that consists of 8 to 200 nucleosides, and wherein the first strand comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142.

In any of the embodiments described herein, miR142 may be miR-142-5p. In any of the embodiments described herein, miR-142 may be miR-142-3p. In some embodiments, the first strand of the oligonucleotide comprises a region that is identical to a seed match region of miR-142-3p or miR-142-5p. In some embodiments, the first strand consists of 8 to 100, 8 to 75.8 to 50, 8 to 40, 8 to 30, 8 to 25, 8 to 23, 8 to 22, 8 to 21, or 8 to 20, 12 to 30, 12 to 25, 12 to 23, 12 to 22, 12 to 21, or 12 to 20 nucleotides. In some embodiments, the oligonucleotide is an siRNA. In some embodiments increasing levels of miR-142 comprises expressing a miR-142 coding sequence in a cell, for example, from a vector. In some embodiments, the miR-142 coding sequence is a coding sequence for pre-miR-142 or pri-miR-142. In some embodiments, the miR-142 coding sequence codes for a shRNA. In some embodiments, a method comprises administering a vector to a subject, or contacting a cell with a vector, wherein the vector comprises a sequence that encodes a shRNA comprising a region that is identical to at least 8, at least 9, at least 10, at least 11, at least 12, at least 13, at least 14, at least 15, at least 16, at least 17, at least 18, at least 19, or at least 20 contiguous nucleotides of miR-142. In some embodiments, a method comprises administering a vector to a subject, or contacting a cell with a vector, wherein the vector comprises a sequence that encodes pre-miR-142.

In some embodiments, the oligonucleotide further comprises a second strand that is complementary to at least a portion of the first strand. In some embodiments, the second strand comprises at least one modified nucleoside.

In some embodiments, a vector comprises a sequence that encodes a pre-miR-142.

In some embodiments, methods of enhancing immune response in a subject are provided. In some embodiments, a method comprises administering to the subject a compound comprising an oligonucleotide, wherein the oligonucleotide consists of 8 to 200 nucleosides, and wherein the oligonucleotide comprises a region that is complementary to at least 8, at least 9, at least 10, at least 11, at least 12, at least 13, at least 14, at least 15, at least 16, at least 17, at least 18, at least 19, or at least 20 contiguous nucleotides of miR-142.

In some embodiments, the subject has cancer. In some embodiments, the cancer is selected from hematologic malignancies and dysplasias such as acute and chronic myeloid leukemia, acute and chronic lymphocytic leukemia, myelodysplasia, Hodgkin's and non-Hodgkin's lymphoma, multiple myeloma and Waldenstrom's macroglobulinemia, myeloproliferative disorders such as myelofibrosis and polycythemia rubra vera; solid tumors such as small-cell and non-small cell lung cancer, breast cancer, colorectal cancer, prostate cancer, ovarian cancer, gastric and esophageal cancer, glioblastoma multiforme, head and neck cancer, pancreatic cancer, hepatocellular carcinoma, soft tissue sarcoma, melanoma, bladder cancer, and renal cancer. In some embodiments, the subject has an infection.

In some embodiments, the infection is an intracellular infection. In some embodiments, the infection is a viral infection, bacterial infection, or parasitic infection.

In some embodiments, the subject has received a vaccine before or at the same time as administration of the compound.

In some embodiments, methods of inhibiting IL-7 receptor signaling in a cell are provided. In some embodiments, a method comprises contacting the cell with a compound comprising an oligonucleotide, wherein the oligonucleotide consists of 8 to 200 nucleosides, and wherein the oligonucleotide comprises a region that is complementary to at least 8, at least 9, at least 10, at least 11, at least 12, at least 13, at least 14, at least 15, at least 16, at least 17, at least 18, at least 19, or at least 20 contiguous nucleotides of miR-142.

In some embodiments, methods of inhibiting IL-7 receptor signaling in a subject with acute lymphoblastic leukemia (ALL) are provided. In some embodiments, a method comprises administering to the subject a compound comprising an oligonucleotide, wherein the oligonucleotide consists of 8 to 200 nucleosides, and wherein the oligonucleotide comprises a region that is complementary to at least 8, at least 9, at least 10, at least 11, at least 12, at least 13, at least 14, at least 15, at least 16, at least 17, at least 18, at least 19, or at least 20 contiguous nucleotides of miR-142.

In some embodiments, methods of reducing regulatory T cell production in a subject are provided. In some embodiments, a method comprises administering to the subject a compound comprising an oligonucleotide, wherein the oligonucleotide consists of 8 to 200 nucleosides, and wherein the oligonucleotide comprises a region that is complementary to at least 8, at least 9, at least 10, at least 11, at least 12, at least 13, at least 14, at least 15, at least 16, at least 17, at least 18, at least 19, or at least 20 contiguous nucleotides of miR-142.

In some embodiments, methods of reducing IgM antibody production in a subject are provided. In some embodiments, a method comprises administering to the subject a compound comprising an oligonucleotide, wherein the oligonucleotide consists of 8 to 200 nucleosides, and wherein the oligonucleotide comprises a region that is complementary to at least 8, at least 9, at least 10, at least 11, at least 12, at least 13, at least 14, at least 15, at least 16, at least 17, at least 18, at least 19, or at least 20 contiguous nucleotides of miR-142.

In any of the embodiments described herein, miR142 may be miR-142-5p. In any of the embodiments described herein, miR-142 may be miR-142-3p. In some embodiments, the oligonucleotide is complementary to at least 8, at least 9, at least 10, at least 11, at least 12, at least 13, at least 14, at least 15, at least 16, at least 17, at least 18, at least 19, or at least 20 contiguous nucleotides of pre-miR-142. In some embodiments, the oligonucleotide comprises a region that is complementary to a seed match region of miR-142-3p or miR-142-5p. In some embodiments, the oligonucleotide consists of 8 to 100, 8 to 75, 8 to 50, 8 to 40, 8 to 30, 8 to 25, 8 to 23, 8 to 22, 8 to 21, 8 to 20, 12 to 30, 12 to 25, 12 to 23, 12 to 22, 12 to 21, or 12 to 20 nucleotides. In some embodiments, the oligonucleotide is a single-stranded oligonucleotide.

In some embodiments, an oligonucleotide comprises at least one modified nucleoside. In some embodiments, at least one modified oligonucleotide comprises a modified sugar moiety, a modified nucleobase moiety, or both. In some embodiments, the oligonucleotide comprises at least one modified internucleoside linkage. In some embodiments, at least one internucleoside linkage is a phosphorothioate linkage. In some embodiments, each internucleoside linkage is a phosphorothioate linkage.

In some embodiments, uses of a compound comprising an oligonucleotide, wherein the oligonucleotide comprises a first strand that consists of 8 to 200 nucleosides, and wherein the first strand comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142 for treating an autoimmune disorder in a subject are provided. In some embodiments, uses of a vector that encodes an shRNA that comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142 for treating an autoimmune disorder in a subject are provided.

In some embodiments, uses of a compound comprising an oligonucleotide, wherein the oligonucleotide comprises a first strand that consists of 8 to 200 nucleosides, and wherein the first strand comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142 for attenuating rejection of a transplanted organ in a subject are provided. In some embodiments, uses of a vector that encodes an shRNA that comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142 for attenuating rejection of a transplanted organ in a subject are provided. In some embodiments, uses of a compound comprising an oligonucleotide, wherein the oligonucleotide consists of 8 to 200 nucleosides, and wherein the oligonucleotide comprises a region that is complementary to at least 8, at least 9, at least 10, at least 11, at least 12, at least 13, at least 14, at least 15, at least 16, at least 17, at least 18, at least 19, or at least 20 contiguous nucleotides of miR-142 for enhancing an immune response in a subject are provided.

In some embodiments, a compound comprising an oligonucleotide, wherein the oligonucleotide comprises a first strand that consists of 8 to 200 nucleosides, and wherein the first strand comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142, for attenuating rejection of a transplanted organ in a subject is provided. In some embodiments, a vector that encodes an shRNA that comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142 for attenuating rejection of a transplanted organ in a subject are provided.

In some embodiments, a compound comprising an oligonucleotide, wherein the oligonucleotide comprises a first strand that consists of 8 to 200 nucleosides, and wherein the first strand comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142, for treating an autoimmune disorder in a subject is provided. In some embodiments, a vector that encodes an shRNA that comprises a region that is identical to at least 8, at least 9, at least 10, at least 11, at least 12, at least 13, at least 14, at least 15, at least 16, at least 17, at least 18, at least 19, or at least 20 contiguous nucleotides of miR-142 for treating an autoimmune disorder in a subject is provided.

In some embodiments, a compound comprising an oligonucleotide, wherein the oligonucleotide consists of 8 to 200 nucleosides, and wherein the oligonucleotide comprises a region that is complementary to at least 8, at least 9, at least 10, at least 11, at least 12, at least 13, at least 14, at least 15, at least 16, at least 17, at least 18, at least 19, or at least 20 contiguous nucleotides of miR-142, for enhancing an immune response in a subject is provided.

Further embodiments and details of the inventions are described below.

### 3. BRIEF DESCRIPTION OF THE FIGURES

FIG. 1A-C: Generation of constitutive and conditional mir-142^{-/-} mice. (a) Schematic showing method of generation of both conditional and constitutive mir-142 deficient mice. ES=embryonic stem cell, neo=neomycin resistance cassette. (b) Schematic (left) and Southern Blot (right) demonstrating validation of successful 5′ recombination in ES cells. (c) Schematic (left) and Southern blot (right) demonstrating validation of successful 3′ recombination in ES cells.

FIG. 2A-C: Genotyping of constitutive mir-142^{-/-} knockout mouse and quantification of mir-142 expression in these mice. (a) Schematic and DNA agarose gel electrophoresis demonstrating specific genotyping of mir-142^{-/-} constitutive knockout mice. (b) Northern Blot demonstrating absence of mir-142 expression in mir-142^{-/-} splenocytes. (c) Quantitative PCR was performed for mir-142-3p and mir-142-5p on RNA isolated from WT, mir-142^{+/-} and mir-142^{-/-} naïve T cells. ND=not detected. Data shown as 2^{-ΔCt} relative to endogenous U6 expression.

FIG. 3: Generation of mir-142^{fl/fl} conditional knockout mouse. Schematic (left) and DNA agarose gel electrophoresis (right) demonstrating specific genotyping of mir-142^{fl} conditional knockout mice.

FIG. 4A-B: Expression of miRNAs in naïve T helper cells and activated T helper cell subsets. (a) Heatmap of miRNA expression in naïve T helper cells and polarised subsets as indicated. miRNAs shown exhibit a fold-change of ≧2.0 between any two conditions. Mean expression of 2 biological replicate arrays is shown. (b) Northern blot analysis demonstrating mir-142-5p expression in the (lane 1-6) 10 bp ladder, total thymocytes, CD4⁺ CD8⁺ DP thymocytes, CD4⁺CD25⁻CD44^{lo}CD62L⁺Thp, AE7 Th1 clone, D10 Th2 clone.

FIG. 5A-F: mir-142 plays a critical role in regulating T cell homeostasis. (a) Flow cytometric analysis of splenocytes from WT and mir-142^{-/-} mice. (b) Individual absolute numbers of CD3⁺ T cells in spleen of mir-142^{-/-}, mir-142^{+/-} and WT littermate mice. (c) Flow cytometric analysis of splenic CD3-gated T cells. Right panel shows CD4:CD8 ratio for 4 independent experiments. (d) Percentage of naïve (CD4⁺CD62L^{high} CD44^{low}CD25⁻) and memory (CD4⁺CD62L^{low}CD44^{high}CD25⁻) and CD4⁺CD62L^{high}CD44^{low}CD25⁺ T cells. (e) Percentage of naïve (CD8⁺CD44^{low}CD122^{low}) and memory (CD8⁺CD44^{high}CD122^{high}) T cells. (f) Immunofluorescence of spleen from WT and mir-142^{-/-} mice stained with anti-CD3 (green), anti-B220 (red) and DAPI nuclear stain (blue). Magnification as indicated. ns=p>0.05 *p<0.05, **p<0.01, ***p<0.001 (unpaired Student's t-test).

FIG. 6: mir-142^{-/-} and WT B cells and dendritic cells. Total cell counts for MHCII⁺CD11c⁺ and CD19⁺ cells. Data from 4 independent experiments.

FIG. 7A-G: The CD4⁺ T cell homeostatic defect in mir-142^{-/-} mice is T cell intrinsic. (a) Sublethally irradiated RAG1 deficient (RAG1^{-/-}) mice were reconstituted intravenously with mir-142^{-/-} or WT bone marrow. Representative plots show analysis of spleens from recipient mice 4 weeks later (left). Percentage CD3⁺ T cells in the spleen of individual recipient mice (right). Mean CD3⁺ percentages±SEM: WT Spleen: 11.8%±3.15%; mir-142^{-/-} Spleen: 0.51%±0.17%; WT LN: 27.1%±2.1%; mir-142^{-/-} LN: 5.2%±0.98%. (b) mir-142^{fl/fl} mice were bred with CD4-Cre transgenic mice. Representative plots show analysis of spleens from CD4-Cre⁺ and CD4-Cre⁻ littermates (left). Right panel shows total numbers of CD3⁺ CD4⁺ T cells in spleen of individual CD4-Cre⁺ and CD4-Cre⁻ littermate mice, **p<0.01 (unpaired Student's t-test). (c) Total thymocyte numbers from individual WT, mir-142^{-/-}, CD4-Cre⁻x mir-142^{fl/fl} and CD4-Cre⁺x mir-142^{fl/fl} mice, ns; p>0.05; WT v mir-142^{-/-} (unpaired Student's t-test), CD4-Cre⁻x mir-142^{fl/fl} v CD4-Cre⁺x mir-142^{fl/fl} mice (Mann-Whitney). (d) Flow cytometric analysis of DN1-4 and ISP (CD8⁺TCR^{lo}HSA⁺CD5^{lo}) thymocytes from individual WT and mir-142 mice (full gating strategy FIG. S5), ns=p>0.05 (unpaired Student's t-test). (e) Representative plots of CD4⁺CD8⁺(DP), CD4⁺ and CD8⁺ thymocytes from WT, mir-142^{-/-}, CD4-Cre⁻x mir-142^{fl/fl} and CD4-Cre⁺x mir-142^{fl/fl} mice. Mean percentage±SEM: WT CD4 SP: 7.7%±0.7%; mir-142^{-/-} CD4 SP: 7.8%±0.5%; CD4-Cre⁻x mir-142^{fl/fl} CD4 SP: 9.9%±0.4%; CD4-Cre⁺x mir-142^{fl/fl} CD4 SP: 6.9%±0.3%; WT CD8 SP: 3.2%±0.4%; mir-142^{-/-} CD8 SP: 2.3%±0.3%; CD4-Cre⁻x mir-142^{fl/fl} CD8 SP: 3.5%±0.06%; CD4-Cre⁺x mir-142^{fl/fl} CD8 SP: 2.4%±0.3%; WT CD4⁺CD8⁺(DP): 77.5%±3.3%; mir-142^{-/-} DP: 74.7%±1.8%; CD4-Cre⁻x mir-142^{fl/fl} DP: 79%±1.3%. CD4-Cre⁺x mir-142^{fl/fl} DP: 86.2%±1.1%. (f) Flow cytometric analysis of total number of DP, TCR^{hi} in the CD4⁺ gate or TCR^{hi} in the CD8⁺gate thymocytes from individual WT, mir-142^{-/-}, CD4-Cre⁻x mir-142^{fl/fl} and CD4-Cre⁺x mir-142^{fl/fl} mice, ns=p>0.05, ** p<0.01; WT v mir-142^{-/-} (unpaired Student's t-test), CD4-Cre⁻x mir-142^{fl/fl} v CD4-Cre⁺x mir-142^{fl/fl} mice (Mann-Whitney). (g) Flow cytometric analysis of TCRγδ⁺ and TCRβ⁺ thymocytes from WT and mir-142^{-/-}. Representative dot plots (left) and results expressed as % of total live thymocytes (middle) and total number (right) from individual mice. ns=p>0.05 (unpaired Student's t-test).

FIG. 8A-E: Thymic flow cytometry gating strategy. (a) Dot plots defining DN1-DN4 thymocytes from WT and mir-142^{-/-} mice on the basis of CD25 and CD44 expression in lin⁻ gate (lin⁻: CD4⁻CD8⁻TCRγδ⁻CD19⁻CD11b⁻CD11c⁻Ly6G⁻NK1.1⁻Ter119⁻). (b) Flow cytometric analysis of DN1-4 and ISP)(CD8⁺TCR^{lo}CD24⁺CD5^{lo}) thymocytes from WT and mir-142^{-/-}. Results expressed as % of total live thymocytes in individual mice. (c) Immature single positive (ISP) thymocyte gating strategy. ISP; CD8⁺TCR^{lo}CD24⁺CD5^{lo}. (d) Gating strategy for single positive (CD4⁺ TCR^{hi} and CD8⁺TCR^{hi}) and double positive (DP; CD4⁺CD8⁺) thymocytes. (e) % total thymocytes that are DP CD4⁺CD8⁺, mature CD4⁺ thymocytes (% CD4⁺ TCRb^{hi} of gated CD4⁺), mature CD8⁺ thymocytes (% CD8⁺TCRb^{hi} of gated CD8⁺) from individual thymii. ns=p>0.05, * p<0.05; WT v mir-142^{-/-} (unpaired Student's t-test), CD4-Cre⁻x mir-142^{fl/fl} v CD4-Cre⁺x mir-142^{fl/fl} mice (Mann-Whitney).

FIG. 9A-F: Naïve mir-142^{-/-} CD4⁺ T cells do not proliferate in vivo. (a) Thp from mir-142^{-/-} and WT were transferred i.p. to RAG1^{-/-}. Spleen and mesenteric lymph nodes (mLN) were analysed 4 weeks later. Representative plots (left) and percentage CD3⁺ T cells in the spleen of individual recipient mice (right), ***=p<0.001 (unpaired Student's t-test). (b) Memory CD4⁺ T cells (CD4⁺CD25⁻CD62L^{low}CD44^{high}) from mir-142^{-/-} and WT were transferred i.p. to RAG1^{-/-}mice. Spleen and mesenteric lymph nodes (mLN) were analysed 4 weeks later. Representative plots (left) and percentage CD3⁺ T cells in the spleen of individual recipient mice (right). (c-f) WT and mir-142^{-/-} Thp were labelled with the cell-tracking reagent CellTrace Violet and transferred intraperitoneally into RAG-1^{-/-} mice. In (c), histograms of CellTrace Violet levels are shown in CD3⁺ CD4⁺ T cells harvested from peritoneal cavity at day 5. In (d), cells were stained with the Pan-Caspase apoptosis detection reagent VAD-FMK-Fitc. Representative FACS histogram (left), percentage VAD-FMK-Fitc⁺in the CD3⁺ CD4⁺ gate in the spleen of individual recipient mice (right), ns=p>0.05 (Mann-Whitney). (e) Total number of CD3⁺ CD4⁺ T cells recovered from peritoneal cavity at day 5 following injection of 2.5×10⁶ Thp into individual RAG1^{-/-} mice. (f) Equal numbers of WT and mir-142^{-/-} Thp were labelled with CFSE and CellTrace Violet, respectively, and co-transferred intraperitoneally into RAG-1^{-/-}. Shown is a representative flow cytometry plot of CD3⁺ CD4⁺ T cells harvested from peritoneal cavity at day 5, mean percentage±SEM (n=3).

FIG. 10A-C: mir-142 deficient CD4⁺ T cells in a T cell transfer model of colitis. (a) Colon weight (left panel) and change in animal weight (right panel) at 4 weeks following i.p. transfer of WT or mir-142^{-/-} Thp. Control mice are RAG1^{-/-}. (b) Representative photograph showing thickening and shortening of colon from RAG1^{-/-} mouse 4 weeks after transfer of WT Thp, compared with mir-142^{-/-} Thp recipient. (c) Representative haematoxylin and eosin stain of colon tissue from RAG1^{-/-} mice that received either WT or mir-142^{-/-} Thp 4 weeks prior to dissection. Sections shown at same magnification. ** p<0.01, *** p<0.001 (unpaired Student's t-test).

FIG. 11A-D: In vitro T cell receptor-mediated activation is unimpaired in the absence of mir-142. (a) mir-142^{-/-} and WT Thp were labelled with CFSE and cultured in Th0 conditions. CFSE staining was assessed at the indicated time points by flow cytometry. Representative histograms (left) and quantification of 4 independent experiments (right). (b) mir-142^{-/-} and WT Thp were cultured in Th0 conditions and stained using Annexin V and propidium iodide (PI). Representative dot plots (left) and quantification of percentage live cells that are Annexin V at the indicated time points from 3 independent experiments (right). (c) mir-142^{-/-} and WT Thp were cultured in vitro in Th0 conditions for 7 days, then transferred intraperitoneally to RAG1^{-/-}. Spleen and mLN were harvested 3 weeks after transfer, representative dot plots are shown (left), percentage CD3 T cells in the spleen of individual recipient mice (right), *=p<0.05 (unpaired Student's t-test). (d) mir-142^{fl/fl} Thp were isolated, transduced with control retrovirus (RV) or Cre-expressing retrovirus and cultured in vitro in Th0 conditions. Both viruses expressed green fluorescent protein (GFP). At day 7, GFP⁺ cells were cell-sorted and injected i.p. into RAG1^{-/-} mice. Spleen and mLN were harvested 3 weeks later. Representative dot plots are shown (left), percentage CD4⁺ T cells in the spleen of individual recipient mice (right).

FIG. 12: Response to varying concentrations of anti-CD3 and anti-CD28 antibody stimulation in mir-142^{-/-} and WT CD4⁺ T cells. mir-142^{-/-} and WT Thp were labelled with CFSE, then stimulated for 4 days in Th0 conditions in plates coated with the indicated concentrations of anti-CD3 and anti-CD28, and analysed by flow cytometry.

FIG. 13: In vitro deletion of mir-142 through transduction of Cre-expressing retrovirus into conditional mir-142^{fl/fl} T cells. DNA agarose gel electrophoresis of PCR genotyping performed on genomic DNA isolated from mir-142^{fl/fl} CD4⁺ T cells that have been transduced with either control retrovirus or Cre-expressing retrovirus (both expressing GFP; GFP⁺ cells flow cytometrically sorted prior to RNA extraction). Shown is genotyping performed with primers for constitutive knockout mice.

FIG. 14A-H: mir-142^{-/-} CD4⁺ T cells are non-responsive to IL-7. (a-b) After 72 h of culture in the presence of IL-7, cells were stained with Annexin V and Aqua Live/Dead reagent. (b) Results from 3 experiments expressed as percentage of Annexin V⁻Live/Dead⁻ after 72 h of culture with or without 10 ng/ml IL-7. ns=p>0.05, ** p<0.01 (unpaired Student's t-test). (c) IL-7Rα and (d) common gamma chain of the IL-7R (IL-2Rγ; CD132) expression in mir-142^{-/-} and WT Thp was analysed using microarray analysis (left panels) and representative flow cytometry histograms (right panels). Microarray analysis data shown as 2^{-ΔCt} relative to β-actin+SEM. Results from 3 experiments, ns=p>0.05, * p<0.05 (unpaired Student's t-test). (e) WT or mir-142^{-/-} Thp were stimulated with 10 ng/ml IL-7 for 30 min on ice and IL-7Rα immunoprecipitates analysed by western blot. Representative blots for expression of IL-7Rα and coprecipitated IL-7Rγ are shown. (f-g) mir-142^{-/-} and WT Thp were cultured in the presence IL-7 at the indicated concentrations. IL-7Rα expression was determined by flow cytometry after 24 h. In (g), results are expressed as change in median fluorescence intensity (MFI) of IL-7Rα following 24 h stimulation with 10 ng/ml IL-7 (n=4), * p<0.05 (Mann-Whitney). (h) Equal numbers of WT and mir-142^{-/-} Thp were differentially labelled and co-transferred into RAG1^{-/-}. IL-7Rα was analysed by flow cytometry after 20 h. Results are representative flow cytometry histograms (left panel) and MFI pre-transfer and 20 h post-transfer (right panel), n=2 for each time point.

FIG. 15: Western blot analysis of pJAK3 in resting mir-142^{-/-} and WT CD4⁺ T cells. (a) Representative western blots for expression of pJAK3 and beta actin from WT or mir-142^{-/-} CD4⁺ Thp whole cell lysates.

FIG. 16A-D: STAT5 phosphorylation is impaired in response to IL-7 in mir-142^{-/-} CD4⁺ T cells. (a) pSTAT levels in the indicated thymocyte populations stimulated with 100 ng/ml IL-7 at time given time intervals. Results are expressed at mean fluorescence intensity (MFI) of pSTAT5 as determined by flow cytometry. (b) CD8⁺ CD44^{low}CD122^{low} sorted T cells from the spleen and lymph nodes of WT or mir-142^{-/-} Thp were stimulated with 100 ng/ml IL-7 at time given time intervals. Results are expressed at mean fluorescence intensity (MFI) of pSTAT5 as determined by flow cytometry. **p<0.01, ns=p>0.05 (unpaired Student's t-test). (c) WT or mir-142^{-/-} Thp were stimulated with 10 ng/ml IL-7 for the indicated time periods. Western blots for expression of pSTAT5, total STAT5 and beta actin are shown. Results are representative of 3 experiments. (d) Intensity of western blot bands were quantified using Genetools. Results are expressed as fold increase above basal pSTAT5 levels. (e) pSTAT5 levels and pSTAT5 nuclear translocation were quantified using ImageStream X. Right panels show percentage of cells positive for pSTAT5 and percentage of cells that have translocated to the nucleus (pSTAT5⁺DAPI⁺) derived from ImageStream data.

FIG. 17A-D: Co-ordinate binding of T-bet and GATA-3 identifies mir-142 as a potential regulator of lineage commitment. (a) Chromatin Immunoprecipitation (ChIP) coupled with massively-parallel sequencing (ChIP-seq) showing occupancy of T-bet, GATA3, histone H3 trimethylated at lysine 4 (H3K4me3) and histone H3 trimethylated at lysine 36 (H3K36me3) at the mir-142 locus (region shown chr17:53,750,000-53,795,000) in human Th1 and Th2 cells, cultured from primary naïve T cells. The number of sequencing reads are plotted per million background-subtracted total reads and aligned with the human genome (hg18). ChIP-Seq data for H3K4me3 and H3K36me3 occupancy in resting CD4+ T-cells are from (19). (b) T-bet occupancy at the mir-142 locus (region shown chr11:87,550,000-87,590,000) in mouse Th1 cells, cultured from primary naïve T cells from wild-type and T-bet^{-/-} mice. The number of sequencing reads are plotted per million background-subtracted total reads and aligned with the mouse genome (mm9) ChIP-Seq data for GATA3 (20) and H3K4me3 and H3K36me3 (21) are shown alongside. (c) Expression of mir-142 in Th1 cells cultured from wild-type and T-bet^{-/-} mice measured by Q-PCR and normalized to U6. (d) Intracellular staining for IFN-γ and IL4 in human in vitro differentiated Th1 cells transduced with using a lentivirus incorporating a green fluorescent protein (GFP) marker or mir-142. Plots are gated on live GFP positive events, and are representative of 3 independent experiments.

FIG. 18: Analysis of ChIP-chip data shows binding of T-bet and GATA-3 at the mir-142 locus in human Th1 and Th2 cells. ChIP was performed on in vitro-polarised human Th1 and Th2 cells, and the presence of binding at promoter regions was determined through the use of tiled promoter microarrays as we have described previously. Jenner et al., 2009, Proc. Natl. Acad. Sci. USA, 106: 17876-17881.

FIG. 19A-H: Default Th1 lineage commitment occurs in the absence of mir-142. (a) Intracellular staining for IFN-γ versus side-scatter (SSC) of Thp cultured in non-polarising Th0 conditions for 7 days. Plots gated on live cells, n=6 mice per group. (b) Percentage of IFN-γ⁺ cells as assessed by flow cytometry. n=6 mice per group, *P<0.05 (Mann Whitney U test). (c) Intracellular staining for IFN-γ versus CFSE in naïve Thp isolated from mir-142^{-/-} and WT littermates cultured in Th0 conditions at the indicated timepoints. Plots representative of 4 independent experiments. (d) mir-142^{-/-} and WT Thp were isolated and cultured in Th0 conditions. Cells were harvested at timepoints throughout the first 72 hours of culture, RNA was extracted and quantitative PCR performed for IFN-γ. The mean of two replicates is shown as 2^{-ΔCt} relative to beta-actin expression. (e) Intracellular staining for 112 versus side-scatter (SSC) of Thp cultured in non-polarising Th0 conditions for 7 days. Plots gated on live cells, representative of 3 independent experiments. (f) Intracellular staining for IFN-γ (vs SSC) of Thp from mir-142^{fl/fl} CD4-Cre⁺ and CD4-Cre⁻ mice cultured in Th0 conditions for 7 days. (g) Intracellular staining for IFN-γ versus T-bet in Thp from mir-142^{fl/fl} mice cultured in Th0 conditions for 7 days. At 24 hours, cells were transduced with retrovirus expressing Cre recombinase and GFP marker, or control retrovirus. Results are representative of 2 independent experiments. (h) Intracellular staining for IFN-γ versus SSC in Thp from mir-142^{-/-} mice cultured in Th0 conditions. At 24 hours, cells were transduced with retrovirus expressing mir-142 and GFP marker or control retrovirus. Results are representative of 3 independent experiments.

FIG. 20: Expression of cytokines under Th0 conditions. WT and mir-142^{-/-} Thp were isolated and cultured in Th0 conditions for 7 days. Intracellular staining was performed for the indicated cytokines. Results representative of 6 independent experiments.

FIG. 21A-C: mir-142 deficient T cells are capable of normal lineage differentiation but exhibit lineage instability and default to IFN-γ production in vivo. (a) Intracellular staining for IFN-γ and IL4 in Thp from mir-142^{-/-} and WT littermate mice cultured in the indicated lineage-skewing conditions for 7 days. Results are representative of 3 independent experiments. (b) Intracellular staining of mir-142^{-/-} and WT Thp initially cultured in either Th2 or Th1 conditions and were then switched at day 3 to the opposing condition for 7 days. i.e. Th2→Th1 means initial Th2 conditions followed by switch to Th1 skewing. Results are representative of 3 independent experiments. (c) Intracellular cytokine staining for IFN-γ and IL17 in Thp harvested from spleen and mesenteric lymph nodes from mir-142^{-/-} or WT littermate mice 4-weeks after adoptive transfer into RAG-1 deficient mice. Results are representative of 2 independent experiments, with 5 mice per group in total.

FIG. 22A-E: mir-142 targets T-bet and controls a negative feedback loop in Th1 lineage commitment. (a) Expression levels of Th1-associated transcription factors in mir-142^{-/-} and WT Thp cultured in vitro for 36 hours in non-polarising conditions. Data shown is the mean of 2 biological replicate arrays. (b) Intracellular staining of T-bet and IFN-γ in WT and mir-142−/− Thp cultured in vitro for 7 days in non-polarising conditions. Results representative of 3 independent experiments. (c) Timecourse analysis of T-bet expression by quantitative PCR in WT and mir-142^{-/-} Thp cultured under Th0 conditions for 72 hours. The mean of two replicates is shown as 2^{-ΔCt} relative to beta-actin expression. (d) Intracellular staining for IFN-γ vs SSC in mir-142^{-/-} Thp cultured in Th0 conditions for 7 days. At 24 hours, cells were transduced with retrovirus expressing a dominant-negative T-bet construct and GFP marker, or with control retrovirus expressing GFP alone. Results are representative of 2 independent experiments.

FIG. 25A-C: CD8+ T cells respond normally to IL-7. (a) expression levels of IL-7Ra in WT and mir-142−/− naïve CD8+ T cells in response to exogenous IL-7. (b) (left) After 72 h of culture in the presence of IL-7, cells were stained with Annexin V and Aqua Live/Dead reagent. (right) Results from 3 experiments expressed as percentage of Annexin V⁻Live/Dead⁻ after 72 h of culture with or without 10 ng/ml IL-7. (c) WT or mir-142^{-/-} CD8+ Thp were stimulated with 10 ng/ml IL-7 for the indicated time periods. Western blots for expression of pSTAT5, total STAT5 and beta actin are shown. Results are representative of 3 experiments. Intensity of western blot bands were quantified using Genetools. Results are expressed as fold increase above basal pSTAT5 levels.

### 4. DETAILED DESCRIPTION

The present inventors have demonstrated that absence of miR-142 in mice disrupts CD8+ T cell development in thymus. A lack of miR-142 also renders peripheral naïve T cells unable to signal through the IL-7 receptor due to an inability to phosphorylate STAT5. Moreover, absence of miR-142 results in a switch to Th1 lineage T cells. These results suggest that miR-142 would be therapeutically beneficial in conditions that would benefit from increased IL-7 receptor signaling, such as autoimmune diseases, and subjects suffering from rejection of transplanted organs. Further, it would be therapeutically beneficial to antagonize miR-142 in conditions that would benefit from a switch to Th1 lineage T cells, such as subjects in need of an enhanced immune response to a vaccine or intracellular infection, or subjects with cancer.

In the sequences herein, “U” and “T” are used interchangeably, such that both letters indicate a uracil or thymine at that position. One skilled in the art will understand from the context and/or intended use whether a uracil or thymine is intended and/or should be used at that position in the sequence. For example, one skilled in the art would understand that native RNA molecules typically include uracil, while native DNA molecules typically include thymine Thus, where a microRNA sequence includes “T”, one skilled in the art would understand that that position in the native microRNA is a likely uracil. Further, synthetic oligonucleotides may comprise U and/or T, and one of ordinary skill in the art can select a suitable nucleobase at each position of the oligonucleotide.

As used herein, “miR-142-5p” refers to a microRNA having the sequence 5′-CAUAAAGUAGAAAGCACUACU-3′ (SEQ ID NO: 1). In some embodiments, the seed match region of miR-142-5p comprises nucleotides 1 to 9, nucleotides 1 to 8, nucleotides 1 to 7, nucleotides 2 to 9, nucleotides 2 to 8, or nucleotides 2 to 7.

As used herein, “miR-142-3p” refers to a microRNA having the sequence 5′-UGUAGUGUUUCCUACUUUAUGGA-3′ (SEQ ID NO: 2). In some embodiments, the seed match region of miR-142-3p comprises nucleotides 1 to 9, nucleotides 1 to 8, nucleotides 1 to 7, nucleotides 2 to 9, nucleotides 2 to 8, or nucleotides 2 to 7.

As used herein, “pre-miR-142” and “miR-142 precursor” refer to a stem-loop having the sequence 5′-GACAGUGCAG UCACCCAUAA AGUAGAAAGC ACUACUAACA GCACUGGAGG GUGUAGUGUU UCCUACUUUA UGGAUGAGUG UACUGUG-3′ (SEQ ID NO: 3).

Unless otherwise indicated, the term “miR-142” encompasses miR-142-5p, miR-142-3p, and pre-miR-142.

As used herein, the term “subject” means a mammal. In some embodiments, a subject is a human.

As used herein, the term “complementary” refers to the ability of a nucleotide on a first nucleic acid to pair with a nucleotide on a second nucleic acid. When a region of a nucleic acid is “complementary” to a region, or set of contiguous nucleotides, of a second nucleic acid, the region may be at least 85%, at least 90%, at least 95%, or 100% complementary to the region, or set of contiguous nucleotides, of the second nucleic acid. Thus, for example, unless otherwise indicated, a region of a first nucleic acid that is complementary to 10 contiguous nucleotides of a second nucleic acid may comprise one mismatch relative to the 10 contiguous nucleotides of the second nucleic acid. An oligonucleotide comprising that region is considered to be complementary to 10 contiguous nucleotides of the second nucleic acid. When there are no mismatches, in some embodiments, the first nucleic acid is said to be “100% complementary” or “fully complementary” to the region, or set of contiguous nucleotides, of the second nucleic acid.

As used herein, the term “oligonucleotide” refers to an oligomer comprising modified and/or unmodified nucleosides. Modified nucleosides may comprise modified sugar moieties and/or modified nucleobase moieties. Further, an oligonucleotide may comprise modified internucleoside linkages, unmodified internucleoside linkages, or both modified and unmodified internucleoside linkages.

4.1. Exemplary Oligonucleotides

In some embodiments, oligonucleotides are provided, wherein the oligonucleotides comprise a region that is complementary to at least 8, at least 9, at least 10, at least 11, at least 12, at least 13, at least 14, at least 15, at least 16, at least 17, at least 18, at least 19, or at least 20 nucleotides of miR-142. In some embodiments, oligonucleotides are provided, wherein the oligonucleotides comprise a region that is complementary to at least 8, at least 9, at least 10, at least 11, at least 12, at least 13, at least 14, at least 15, at least 16, at least 17, at least 18, at least 19, or at least 20 nucleotides of miR-142-5p. In some such embodiments, the region is also complementary to at least 8, at least 9, at least 10, at least 11, at least 12, at least 13, at least 14, at least 15, at least 16, at least 17, at least 18, at least 19, or at least 20 nucleotides of pre-miR-142.

In some embodiments, oligonucleotides are provided, wherein the oligonucleotides comprise a region that is complementary to at least 8, at least 9, at least 10, at least 11, at least 12, at least 13, at least 14, at least 15, at least 16, at least 17, at least 18, at least 19, or at least 20 nucleotides of miR-142-3p. In some such embodiments, the region is also complementary to at least 8, at least 9, at least 10, at least 11, at least 12, at least 13, at least 14, at least 15, at least 16, at least 17, at least 18, at least 19, or at least 20 nucleotides of pre-miR-142.

In some embodiments, oligonucleotides are provided, wherein the oligonucleotides comprise a region that is complementary to at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 70, or at least 80 nucleotides of pre-miR-142.

In some embodiments, an oligonucleotide that comprises a region that is complementary to miR-142 is a single-stranded oligonucleotide. In some embodiments, an oligonucleotide that comprises a region that is complementary to miR-142 is referred to as a miR-142 antagonist or a miR-142 antisense. Single-stranded microRNA inhibitors are commercially available, for example, from Qiagen (miScript miRNA inhibitors), Life Technologies (Ambion® Anti-miR™ miRNA inhibitors), and Exiqon (miRCURY LNA™ microRNA inhibitors). In some embodiments, the oligonucleotide that comprises a region that is complementary to miR-142 is a hairpin microRNA inhibitor, which comprises a self-complementary region such that the oligonucleotide folds into a hairpin. Commercial microRNA hairpin inhibitors are available, e.g., from Thermo Scientific (miRIDIAN microRNA hairpin inhibitors).

In some embodiments, oligonucleotides are provided, wherein the oligonucleotides comprise a region that is identical to at least 8, at least 9, at least 10, at least 11, at least 12, at least 13, at least 14, at least 15, at least 16, at least 17, at least 18, at least 19, or at least 20 nucleotides of miR-142. In some embodiments, oligonucleotides are provided, wherein the oligonucleotides comprise a region that is identical to at least 8, at least 9, at least 10, at least 11, at least 12, at least 13, at least 14, at least 15, at least 16, at least 17, at least 18, at least 19, or at least 20 nucleotides of miR-142-5p. In some such embodiments, the region is also identical to at least 8, at least 9, at least 10, at least 11, at least 12, at least 13, at least 14, at least 15, at least 16, at least 17, at least 18, at least 19, or at least 20 nucleotides of pre-miR-142.

In some embodiments, oligonucleotides are provided, wherein the oligonucleotides comprise a region that is identical to at least 8, at least 9, at least 10, at least 11, at least 12, at least 13, at least 14, at least 15, at least 16, at least 17, at least 18, at least 19, or at least 20 nucleotides of miR-142-3p. In some such embodiments, the region is also identical to at least 8, at least 9, at least 10, at least 11, at least 12, at least 13, at least 14, at least 15, at least 16, at least 17, at least 18, at least 19, or at least 20 nucleotides of pre-miR-142.

In some embodiments, oligonucleotides are provided, wherein the oligonucleotides comprise a region that is identical to at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 70, or at least 80 nucleotides of pre-miR-142.

In some embodiments, an oligonucleotide that comprises a region that is identical to miR-142 is a miR-142 mimic. In some such embodiments, the oligonucleotide may further comprise a complementary strand. In some such embodiments, the oligonucleotide may be referred to as a double-stranded miR-142 mimic. Double-stranded microRNA mimics are commercially available, e.g., from Qiagen (miScript), Sigma Aldrich, Invitrogen (mirVana), and Thermo Scientific (miRIDIAN microRNA mimics). In some embodiments, the strand comprising the region that is identical to miR-142 and the complementary strand are part of a single oligonucleotide. In some embodiments of double-stranded miR-142 mimics, the double-stranded region of the mimic is 15 to 30 nucleotides in length.

In some embodiments, a miR-142 mimic is a siRNA. A siRNA, or small interfering RNA, is an RNA comprising a double-stranded region of 15 to 25 base pairs in length. In some embodiments, an siRNA comprises a 5′-phosphate and a 3′-hydroxyl. In some embodiments, an siRNA comprises a two-base overhang (i.e., single-stranded region) on the 3′ end of one or both of the RNA strands. One strand of the siRNA is an oligonucleotide (such as an RNA oligonucleotide) that comprise a region that is identical to at least 8, at least 9, at least 10, at least 11, at least 12, at least 13, at least 14, at least 15, at least 16, at least 17, at least 18, at least 19, or at least 20 nucleotides of miR-142. The oligonucleotide, in some embodiments, comprises a region that is identical to the seed match region of miR-142. In some embodiments, such an siRNA targets the same site(s) in the same genes as miR-142, and is therefore considered to be a mimic.

In some embodiments, a miR-142 mimic is a shRNA. A shRNA, or short hairpin RNA, comprises a single RNA strand that is self-complementary over at least a portion of the RNA. In some embodiments, an shRNA is delivered to a subject by administering a vector comprising a coding sequence for the shRNA, such as in gene therapy. See, e.g., Xiang et al., 2006, Nature Biotech., 24: 697-702; Senzer et al., 2012, Mol. Therap., 20: 679-686; US 20100299771; US 20120004283. In some embodiments, a vector encoding a shRNA may be administered in order to increase miR-142 levels in a particular cell, cell type, tissue, or subject. In some embodiments, a vector comprising the coding sequence for the miR-142 pre-miRNA or pri-miRNA may be administered in order to increase miR-142 levels in a particular cell, cell type, tissue, or subject.

4.1.1. Exemplary Oligonucleotide Modifications

In some embodiments, an oligonucleotide comprises at least one modified nucleoside and/or modified internucleoside linkage. In some embodiments, such modifications may increase the binding affinity and specificity of an oligonucleotide for its target nucleic acid as compared to oligonucleotides that contain only deoxyribonucleotides, and may allow for the use of shorter polynucleotides or for shorter regions of complementarity between the oligonucleotide and the target nucleic acid. In some embodiments, such modifications may (or may also) increase the nuclease resistance of the oligonucleotide, improving the pharmacokinetics such that lower doses of the oligonucleotide may be needed to therapeutic effect.

In some embodiments, an oligonucleotide includes one or more modified nucleosides, wherein each modified nucleoside comprises a modified nucleobase moiety and/or a modified sugar moiety. In some embodiments, an oligonucleotide comprises one or more modified internucleoside linkages, one or more unmodified internucleoside linkages, or a combination of modified and unmodified internucleoside linkages.

Nonlimiting exemplary modified nucleosides having modified nucleobase moieties include nucleosides comprising 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine. Nonlimiting exemplary

Nonlimiting exemplary modified nucleosides having modified sugar moieties include nucleosides comprising 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars, and bicyclic sugars, such as locked nucleic acid (“LNA”). In some embodiments, modified sugars are arabinose sugars, or d-arabino-hexitol sugars.

In some embodiments, an oligonucleotide comprises one or more backbone modifications such as peptide nucleic acids (PNA; e.g., an oligomer including nucleobases linked together by an amino acid backbone). Other backbone modifications include, but are not limited to, phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

One skilled in the art can design a suitable oligonucleotide for an intended application using the knowledge in the art. Nonlimiting exemplary descriptions of oligonucleotides that antagonize microRNAs or mimic microRNAs (such as siRNA) and considerations for designing such oligonucleotides, including suitable modified nucleosides and internucleoside linkages, are described, for example, in U.S. Publication No. 20110166198; U.S. Pat. No. 8,017,763; U.S. Pat. No. 8,173,611; WO 2005013901; US 2012-0184596; US 2009270481; EP 1984382; EP1824975; U.S. Pat. No. 7,834,170; WO 2012149646; Breving et al. Int J Biochem Cell Biol. 2010 August; 42(8):1316-29; van Rooij et al., Circ Res. 2012 Feb. 3; 110(3):496-507; Iorio et al., EMBO Mol. Med. 2012 March; 4(3): 143-159; Frieden, M. et al. (2008) Curr. Pharm. Des. 14(11):1138-1142, each of which is incorporated by reference herein in its entirety for any purpose.

4.2. Methods

In some embodiments, methods of treating autoimmune diseases are provided. In some such embodiments, a method comprises administering to a subject with an autoimmune disease a miR-142 mimic. In some embodiments, a miR-142 mimic is a compound comprising an oligonucleotide, wherein the oligonucleotide comprises a first strand that consists of 8 to 200 nucleosides, and wherein the first strand comprises a region that is identical to at least 8 contiguous nucleotides of miR-142. Nonlimiting exemplary autoimmune conditions that may be treated using the methods described herein include rheumatoid arthritis, multiple sclerosis, psoriasis, and inflammatory bowel disease.

In some embodiments, methods of attenuating rejection of a transplanted organ are provided. In some such embodiments, a method comprises administering to a subject who has undergone an organ transplant a miR-142 mimic. In some embodiments, a miR-142 mimic is a compound comprising an oligonucleotide, wherein the oligonucleotide comprises a first strand that consists of 8 to 200 nucleosides, and wherein the first strand comprises a region that is identical to at least 8 contiguous nucleotides of miR-142. In some embodiments, the method comprises administering to the subject a vector that encodes a miR-142 or miR-142 mimic, such as a shRNA, a pre-miR-142, or a pri-miR-142. As used herein, “attenuating rejection” includes delaying the onset of organ rejection and/or lessening the severity of organ rejection. In some embodiments, the subject has received a transplanted kidney, liver, lung, bone marrow, limb (such as hand) and/or heart.

In some embodiments, methods of enhancing IL-7 receptor signaling in a cell are provided. In some such embodiments, a method comprises contacting a cell with a miR-142 mimic. In some embodiments, a miR-142 mimic is a compound comprising an oligonucleotide, wherein the oligonucleotide comprises a first strand that consists of 8 to 200 nucleosides, and wherein the first strand comprises a region that is identical to at least 8 contiguous nucleotides of miR-142. In some embodiments, the method comprises contacting the cell with a vector that encodes a miR-142 or miR-142 mimic, such as a shRNA, a pre-miR-142, or a pri-miR-142. In some embodiments, the cell is in a subject. In some such embodiments, the subject is infected with HIV. Thus, in some embodiments, methods of treating subjects with HIV are provided, comprising administering to the subject a miR-142 mimic. In some embodiments, the method comprises administering to the subject a vector that encodes a miR-142 or miR-142 mimic, such as a shRNA, a pre-miR-142, or a pri-miR-142. In some embodiment, a miR-142 mimic is a compound comprising an oligonucleotide, wherein the oligonucleotide comprises a first strand that consists of 8 to 200 nucleosides, and wherein the first strand comprises a region that is identical to at least 8 contiguous nucleotides of miR-142.

In some embodiments, methods of increasing regulatory T cell production in a subject are provided. In some such embodiments, a method comprises administering to the subject a miR-142 mimic. In some embodiments, the method comprises administering to the subject a vector that encodes a miR-142 or miR-142 mimic, such as a shRNA, a pre-miR-142, or a pri-miR-142. In some embodiments, a miR-142 mimic is a compound comprising an oligonucleotide, wherein the oligonucleotide comprises a first strand that consists of 8 to 200 nucleosides, and wherein the first strand comprises a region that is identical to at least 8 contiguous nucleotides of miR-142.

In some embodiments, methods of increasing IgM production in a subject are provided, wherein the methods comprise administering to the subject a miR-142 antagonist. In some embodiments, a miR-142 antagonist is a compound comprising an oligonucleotide, wherein the oligonucleotide comprises a first strand that consists of 8 to 200 nucleosides, and wherein the first strand comprises a region that is identical to at least 8 contiguous nucleotides of miR-142.

In any of the embodiments of miR-142 mimic, the oligonucleotide may comprise a second strand, for example, when the oligonucleotide is a double-stranded miR-142 mimic. In some embodiments, the oligonucleotide is a hairpin, wherein the first strand and the second strand are part of a single contiguous oligonucleotide.

In any of the embodiments described herein, miR-142 may be miR-142-5p and/or miR-142-3p. Further, the region of the oligonucleotide first strand that is identical to miR-142 may be identical to at least 8, at least 9, at least 10, at least 11, at least 12, at least 13, at least 14, at least 15, at least 16, at least 17, at least 18, at least at least 19, or at least 20 contiguous nucleotides of miR-142. In some embodiments, when miR-142 is miR-142-3p, the first strand is identical to the entire miR-142-3p sequence (23 contiguous nucleotides, SEQ ID NO: 2). In some embodiments, when miR142 is miR-142-5p, the first strand is identical to the entire miR-142-5p sequence (22 contiguous nucleotides, (SEQ ID NO: 1).

In some embodiments, the oligonucleotide first strand comprises a region that is identical to a seed match region of miR-142-3p or miR-142-5p. In some embodiments, the seed match region of miR-142-3p is nucleotides 1 to 9, nucleotides 1 to 8, nucleotides 1 to 7, nucleotides 2 to 9, nucleotides 2 to 8, or nucleotides 2 to 7 of miR-142-3p (SEQ ID NO: 2). In some embodiments, the seed match region of miR-142-5p is nucleotides 1 to 9, nucleotides 1 to 8, nucleotides 1 to 7, nucleotides 2 to 9, nucleotides 2 to 8, or nucleotides 2 to 7 of miR-142-5p (SEQ ID NO: 1). In various embodiments, the first strand of the oligonucleotide may consist of 8 to 100, 8 to 75, 8 to 50, 8 to 40, 8 to 30, 8 to 25, 8 to 23, 8 to 22, 8 to 21, or 8 to 20, 12 to 30, 12 to 25, 12 to 23, 12 to 22, 12 to 21, or 12 to 20 nucleotides.

In any of the embodiments described herein, a vector may encode a shRNA comprising a region that is identical to miR-142 may be identical to at least 8, at least 9, at least 10, at least 11, at least 12, at least 13, at least 14, at least 15, at least 16, at least 17, at least 18, at least at least 19, or at least 20 contiguous nucleotides of miR-142. In any of the embodiments described herein, a vector may encode a sequence comprising pre-miR-142 or pri-miR-142. In some such embodiments, the vector expresses the pre-miR-142 or pri-miR-142, which is then processed by the cell to produce miR-142-5p and/or miR-142-3p. Thus, in some embodiments, a method comprises increasing levels of miR-142-3p and/or miR-142-5p, for example, by contacting a cell with a vector that encodes the pre-miR-142 or pri-miR-142.

In some embodiments, methods of enhancing an immune response in a subject are provided. In some such embodiments, a method comprises administering to the subject a miR-142 antagonist. In some embodiments, a miR-142 antagonist is a compound comprising an oligonucleotide, wherein the oligonucleotide consists of 8 to 200 nucleosides, and wherein the oligonucleotide comprises a region that is complementary to at least 8 contiguous nucleotides of miR-142. In some embodiments, the method comprises enhancing an immune response in a subject with cancer. Nonlimiting exemplary cancers include hematologic malignancies and dysplasias such as acute and chronic myeloid leukemia, acute and chronic lymphocytic leukemia, myelodysplasia, Hodgkin's and non-Hodgkin's lymphoma, multiple myeloma and Waldenstrom's macroglobulinemia, myeloproliferative disorders such as myelofibrosis and polycythemia rubra vera; solid tumors such as small-cell and non-small cell lung cancer, breast cancer, colorectal cancer, prostate cancer, ovarian cancer, gastric and esophageal cancer, glioblastoma multiforme, head and neck cancer, pancreatic cancer, hepatocellular carcinoma, soft tissue sarcoma, melanoma, bladder cancer, and renal cancer. In some embodiments, the method comprises enhancing an immune response in a subject with an infection. In some such embodiments, the subject has an intracellular infection, such as a virus or tuberculosis. Further nonlimiting exemplary infections include herpes virus infections (CMV, EBV, HSV 1+2) and other viral infections such as influenza, rhinovirus, echovirus, and HIV; and certain bacterial infections such as listeria, brucella, legionella, francisella; and intracellular parasites, such as chlamydia, rickettsia.

In some embodiments, methods of enhancing an immune response to a vaccine are provided. In some such embodiments, a method comprises administering to the subject a miR-142 antagonist. In some embodiments, a miR-142 antagonist is a compound comprising an oligonucleotide, wherein the oligonucleotide consists of 8 to 200 nucleosides, and wherein the oligonucleotide comprises a region that is complementary to at least 8 contiguous nucleotides of miR-142. The vaccine may be administered prior to administration of the miR-142 antagonist (i.e., more than 4 hours before, more than 1 day, more than 2 days, more than 4 days, more than 1 week, or more than 2 weeks before), contemporaneously with the miR-142 antagonist (i.e., within 4 hours before or after administration of the miR-142 antagonist), or after the miR-142 antagonist (i.e., more than 4 hours, more than 1 day, more than 2 days, more than 4 days, more than 1 week, or more than 2 weeks after).

In some embodiments, methods of inhibiting IL-7 receptor signaling are provided. In some such embodiments, a method comprises contacting a cell with a miR-142 antagonist. In some embodiments, a miR-142 antagonist is a compound comprising an oligonucleotide, wherein the oligonucleotide consists of 8 to 200 nucleosides, and wherein the oligonucleotide comprises a region that is complementary to at least 8 contiguous nucleotides of miR-142. In some embodiments, methods of treating acute lymphoblastic leukemia (ALL) are provided. In some such embodiments, a method comprises administering to the subject a miR-142 antagonist. In some embodiments, a miR-142 antagonist is a compound comprising an oligonucleotide, wherein the oligonucleotide consists of 8 to 200 nucleosides, and wherein the oligonucleotide comprises a region that is complementary to at least 8 contiguous nucleotides of miR-142. In some embodiments, the ALL comprises hyperactivated IL-7 receptor signaling. In some embodiments, the ALL comprises an activating mutation in IL-7 receptor.

In some embodiments, methods of reducing regulatory T cell production are provided. In some such embodiments, a method comprises administering to the subject a miR-142 antagonist. In some embodiments, a miR-142 antagonist is a compound comprising an oligonucleotide, wherein the oligonucleotide consists of 8 to 200 nucleosides, and wherein the oligonucleotide comprises a region that is complementary to at least 8 contiguous nucleotides of miR-142.

In some embodiments, methods of reducing IgM antibody production are provided. In some such embodiments, a method comprises administering to the subject a miR-142 antagonist. In some embodiments, a miR-142 antagonist is a compound comprising an oligonucleotide, wherein the oligonucleotide consists of 8 to 200 nucleosides, and wherein the oligonucleotide comprises a region that is complementary to at least 8 contiguous nucleotides of miR-142.

In any of the embodiments of a miR-142 antagonist herein, the miR-142 may be miR-142-3p, miR-142-5p, or pre-miR-142. In some embodiments, the oligonucleotide comprises a region that is identical to at least 8, at least 9, at least 10, at least 11, at least 12, at least 13, at least 14, at least 15, at least 16, at least 17, at least 18, at least at least 19, or at least 20 contiguous nucleotides of miR-142. In some embodiments, the oligonucleotide comprises a region that is complementary to a seed match region of miR-142-3p or miR-142-5p. In some embodiments, the seed match region of miR-142-3p is nucleotides 1 to 9, nucleotides 1 to 8, nucleotides 1 to 7, nucleotides 2 to 9, nucleotides 2 to 8, or nucleotides 2 to 7 of miR-142-3p (SEQ ID NO: 2). In some embodiments, the seed match region of miR-142-5p is nucleotides 1 to 9, nucleotides 1 to 8, nucleotides 1 to 7, nucleotides 2 to 9, nucleotides 2 to 8, or nucleotides 2 to 7 of miR-142-5p (SEQ ID NO: 1). In some embodiments, the miR-142 antagonist comprises a single-stranded oligonucleotide.

In various embodiments, oligonucleotide comprises at least one modified nucleoside and/or at least one modified internucleoside linkage. In some embodiments, a modified nucleoside comprises a modified sugar moiety and/or a modified nucleobase moiety. Nonlimiting exemplary modified nucleosides are known in the art. In some embodiments, a modified internucleoside linkage is phosphorothioate. In some embodiments, each internucleoside linkage in the oligonucleotide are phosphorothioate. Nonlimiting exemplary modified nucleosides and modified internucleoside linkages are known in the art.

4.3. Pharmaceutical Compositions

In some embodiments, a pharmaceutical composition is formulated and administered according to Semple et al., Nature Biotechnology advance online publication, 17 Jan. 2010 (doi:10.1038/nbt.1602)), which is incorporated by reference herein in its entirety for any purpose.

The terms “treat,” “treating” and “treatment” as used herein refer to ameliorating symptoms associated with cancer, including preventing or delaying the onset of symptoms and/or lessening the severity or frequency of symptoms of the cancer.

The term “effective amount” of a target RNA or an inhibitor of target RNA expression or activity is an amount sufficient to treat the condition. An effective amount of a compound for use in the pharmaceutical compositions disclosed herein may be determined by a person skilled in the art, e.g., by taking into account factors such as the size and weight of the individual to be treated, the stage of the disease, the age, health and gender of the individual, the route of administration and whether administration is localized or systemic.

In addition to the oligonucleotides described herein, or a pharmaceutically acceptable salts thereof, the pharmaceutical compositions disclosed herein may further comprise a pharmaceutically acceptable carrier, including but not limited to, water, buffered water, normal saline, 0.4% saline, 0.3% glycine, and hyaluronic acid. In some embodiments, the pharmaceutical compositions comprising an oligonucleotide may be encapsulated, e.g., in liposomes, such as stable nucleic acid-lipid particles (SNALPs). See, e.g., Owens, Nat. Rev. Drug Discov., 2005, 4:717 and references cited therein; Geisbert et al., Lancet, 2010, 375: 1896-905; de Antonellis et al., Naunyn Schmiedebergs Arch Pharmacol., 2013, PMID: 23354452. In some embodiments, the pharmaceutical compositions further comprise pharmaceutically acceptable excipients such as stabilizers, antioxidants, osmolality adjusting agents and buffers.

Pharmaceutical compositions can take the form of solutions, suspensions, emulsions, tablets, pills, pellets, capsules, capsules containing liquids, powders, sustained-release formulations, suppositories, emulsions, aerosols, sprays, suspensions, or any other form suitable for use. Methods of administration include, but are not limited to, oral, parenteral, intravenous, oral, and by inhalation.

Descriptions of nonlimiting exemplary formulations for compounds comprising oligonucleotides are described, for example, in US 20110166198; US 20120183581; US 20110091525; US 20110060032; Owens, Nat. Rev. Drug Discov., 2005, 4:717 and references cited therein; Geisbert et al., Lancet, 2010, 375: 1896-905; de Antonellis et al., Naunyn Schmiedebergs Arch Pharmacol., 2013, PMID: 23354452.

The following examples are for illustration purposes only, and are not meant to be limiting in any way.

### 5. EXAMPLES

### 5.1 Example 1

### Materials and Methods

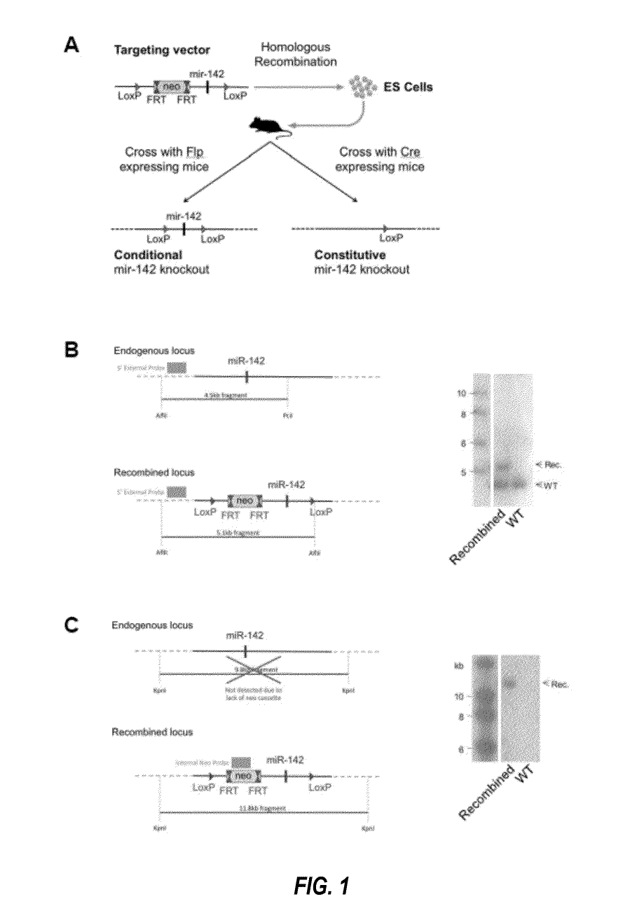

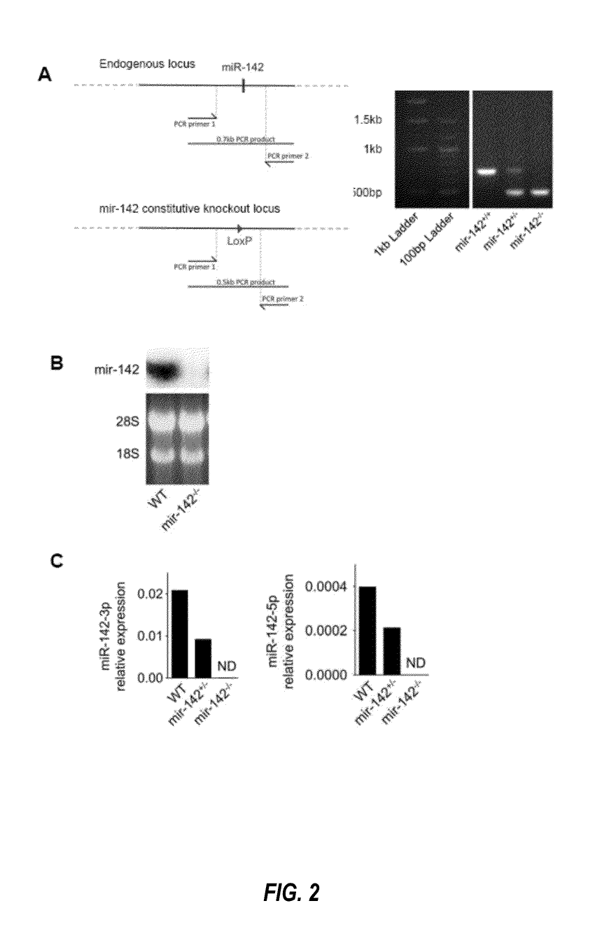

Generation of mir-142 deficient mice. Mice were generated by homologous recombination in 129Sv mouse embryonic stem cells using a targeted vector conferring neomycin resistance. This vector contained both loxP and FRT sites flanking the mir-142 locus and neomycin-resistance cassette in such a way that both conditional and constitutive mir-142 deficient mice could be generated (FIGS. 1 to 3). Chimeric offspring were then bred with C57BL/6J-Cre deleter mice to generate mice carrying the constitutive mir-142 knockout allele, or the C57BL/6J-Flp deleter mice to generate conditional mir-142 deficient mice (FIG. 2). Marker-Assisted Accelerated Backcrossing (MAXBAX, Charles River) sequencing showed that the mice were 89-95% C57B16. WT controls are mir-142^{+/+} littermates. All experimental protocols involving rodents were reviewed and approved by our local ethics review committee and were carried out in accordance with a UK Home Office Project Licence (License #: PPL706792).

Naïve T Cell Isolation and In Vitro Culture.

CD4⁺ T cells were isolated from mouse lymph nodes and spleen using CD4 microbeads (Miltenyi Biotec). Cells were then labelled with fluorochrome-conjugated antibodies to CD4, CD62L, CD44 and CD25 (eBioscience). Naïve T cells were sorted using a BD FACSAria II flow cytometric cell sorter (Becton Dickinson) to >98% purity. Cells were initially activated for three days with plate-bound anti-CD3 (2 μg/ml) and anti-CD28 (2 μg/ml) antibodies (Bio X Cell), and cultured for a total of seven days in 10% fetal calf serum-supplemented RPMI-1640 cell culture medium (PAA) under non-polarizing conditions in the presence of IL-2 (20 ng/ml, R&D Systems). For IL-7 experiments, cells were cultured in culture medium supplemented with 10 ng/ml (unless otherwise indicated) recombinant mouse IL-7 (R&D Systems).

Bone Marrow Transfer.

RAG-1 deficient mice were sublethally irradiated with 3.5 Gy from a Caesium-137 source. Bone marrow (BM) was isolated by flushing donor mouse femur and tibia with sterile phosphate buffered saline (PBS), and then mechanically disrupting this suspension through sterile mesh. 1×10⁶ BM cells were injected intravenously via the tail vein immediately after irradiation. Mice were monitored for weight loss and signs of disease throughout the course of the experiment.

T Cell Transfer.

RAG-1^{-/-} mice were injected with cells resuspended in sterile PBS. Mice were weighed prior to injection and monitored for weight loss and signs of disease onset including diarrhoea, rectal bleeding, weight loss and for signs of peritonism. For naïve T cell transfer, 0.5×10⁶ cells were injected; for transfer of in vitro activated T cells: 1×10⁶; for short-term (5 day) in vivo tracking experiments: 2.5×10⁶.

Flow Cytometry.

The following anti-mouse antibodies were used for flow cytometry; CD3 (145-2C11), CD4 (RM4.5), CD8 (53-6.7), CD62L (MEL-14), CD44 (IM7), CD25 (PC61.5), CD19 (1D3), CD11c (N418), CD127 (A7R34), CD132 (TUGh4), TCRβ (H57-597), TCRγδ (G13). Phosphorylated STAT5 was stained using a BD Phosflow kit according to manufacturer's instructions and acquired using an ImageStream X (Amnis). For CFSE and CellTrace Violet (both Invitrogen) tracking experiments, cells were isolated and then labelled according to the manufacturer's protocol with either CFSE (2 μM) or CellTrace Violet (2 μM). In vitro apoptosis staining was performed according to the manufacturers' instructions with Annexin V-Pacific Blue (eBioscience) and Propidium Iodide (Invitrogen). Ex vivo apoptosis staining was performed using VAD-FMK-Fitc (Promega). Live/Dead staining was performed with Live/Dead Yellow or Live/Dead Aqua (Invitrogen). Samples were acquired using BD LSR II and Fortessa flow cytometers (Becton Dickinson). Data were analysed with FlowJo software (Treestar, USA).

BrdU Labelling.

Each mouse received one i.p. injection of 1 mg BrdU and then further exposed to BrdU (0.8 mg/ml) in their drinking water thereafter for up to 8 days. Thymocytes were harvested and stained using a BrdU flow kit according to the manufacturer's instructions (BD Pharmingen) and analysed by flow cytometry.

Microarrays.

Total RNA was extracted using Trizol (Invitrogen). Protein-coding gene microarrays were performed at the King's College London Genomics Centre facility. RNA was labelled and hybridized to the Affymetrix Mouse Gene ST 1.0 microarray using Nugen WT-Ovation Pico (Nugen Technologies) and Affymetrix sample preparation kits according to the manufacturer's instructions. Two biological replicates were performed for each genotype. Data were normalized using the robust multi-array average (RMA) method³⁷. miRNA microarrays were performed using a Cepheid Inc. (Maurens-Scopont, France) custom microRNA microarray platform spotted with probes designed to detect miRNAs included in miRBase release 11 (April 2008). RNA was size-fractionated to the <40 nt fraction using the Ambion FlashPAGE fractionator (Life Technologies Inc., Carlsbad, Calif.). RNA was 3′ labelled by overnight ligation of a UUUU-Cy5 fluorescent tag. Labelled RNA was hybridized overnight to pre-prepared microarray slides, and fluorescence was then measured using the GenePix 4000A scanner in conjunction with GenePix Pro Software (Molecular Devices, Sunnyvale, Calif., USA). Two biological replicates were performed for each genotype. Data were normalized using the vsn bioconductor package³⁸, and analysed with the MayDay software analysis package³⁹.

Real-Time RT-PCR.

mRNA and miRNA expression was quantified using Taqman assays (Applied Biosystems). Reactions were performed according to the manufacturer's instructions and analysed using a 7500 real-time PCR instrument (Applied Biosystems).

Northern Blot.

Northern blot was performed for the detection of miR-142-3p and miR-142-5p RNA. Total RNA was isolated using Trizol. 40 μg of total RNA in RNA sample loading buffer (Sigma Aldrich) was loaded into each well of a 1% denaturing formaldehyde-agarose gel. Following electrophoresis, RNA was transferred onto Hybond-N+ membrane (GE Healthcare UK Ltd., Little Chalfont, UK). Chemiluminescent miRNA Northern Blot kits (Signosis Inc., Sunnyvale, Calif., USA) specific for miR-142-3p and miR-142-5p were used for detection of miRNA, according to manufacturer's instructions. Briefly, hybridization was performed by overnight incubation under rotation at 42° C. in the presence of biotin-labelled miRNA probes. After washing, membrane was incubated with streptavidin-HRP, then washed again, and finally incubated with detection substrate. Detection was performed with the Hyperfilm imaging system (GE Healthcare).

Western Blot.

Cells were sorted as described above, and rested in serum free medium for 2 h. Cell suspensions were cultured with 10 ng/ml recombinant mouse IL-7, washed in PBS and lysed in RIPA buffer. Samples were boiled in reducing sample buffer and protein separated using SDS-PAGE (Biorad) before transfer to a nitrocellulose membrane. Blots were probed with rabbit anti-mouse STAT5, pSTAT5, pJAK3, and β-actin (Cell Signalling). For immunoprecipitation equal numbers of Thp were incubated for 30 minutes on ice with rmIL-7. Whole cell lysates were prepared in RIPA buffer, incubated with anti-CD127 coated DynaBeads beads and magnetically separated. Lysates were separated by SDS/PAGE under reducing conditions. After transfer to a nitrocellulose membrane, blocking was performed with Tris buffered saline containing 5% (vol/vol) skim milk. Blots were then incubated with anti-CD127 and anti-CD132 overnight HRP-conjugated goat anti-rabbit IgG or rabbit anti-goat IgG were used for secondary detection (GE Healthcare). Blots were developed using enhanced chemiluminescence (Pierce Biotechnology).

Immunohistochemistry.

Splenic tissue was removed, snap frozen in Jung tissue freezing medium (Leica Microsystems Nussloch GmbH). Cryostat sections were fixed in acetone before blocking with 20% normal horse serum (PAA Laboratories Inc.). Sections were incubated with FITC-conjugated anti-CD3 (eBioscience Ltd), biotin-conjugated anti-B220 (eBioscience Ltd), followed by streptavidin-conjugated alexa594 (Invitrogen). Nuclei were visualised by staining with 1 μg/ml DAPI (Invitrogen). Images were acquired on an Olympus BX51 microscope using Micro-Manager software (Vale Laboratory).

Retroviral Transfections and Transductions.

Human 293T cells were used as a packaging cell line to generate retroviral stocks for transduction by conventional techniques. For transduction, CD3CD28 activated CD4 cells were seeded at 1×10⁶ cells per well of a 48 w plate and maintained in DMEM+10% FCS+20 ng/ml rhIL-2 for 36 hours. At transduction 8 μM polybrene and viral supernatant were added and centrifuged at 2500 rpm for 90 min at 22° C. Medium was replaced after a further 12 h and GFP cell sorted using a BD FACSAria II flow cytometric cell sorter (Becton Dickinson).

### 5.2 Example 2

### Mir-142 Controls Naïve T Cell Homeostasis In Vivo

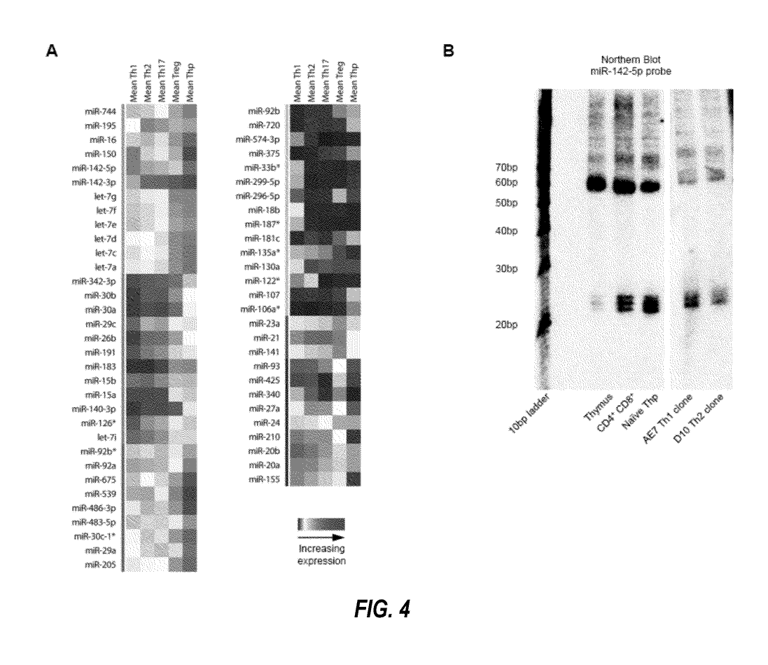

In order to identify miRNAs of importance in CD4⁺ T cell homeostatic control we initially performed microarray profiling of miRNA expression in naïve CD4⁺CD25⁻CD62L^{high} CD44^{low} T cells (Thp) and highly polarised T cell subsets derived from these cells (FIG. 4). Bioinformatic analysis demonstrated a group of miRNAs that were highly expressed in the naïve state, but subsequently down-regulated upon differentiation (FIG. 4a). Of these, mir-142-3p and mir-142-5p were among the most highly expressed in Thp cells. Analysis using Northern blot confirmed the dynamic regulation of mir-142 expression between the naïve and T helper cell subsets (FIG. 4b), suggesting a potential function of mir-142 in the regulation of processes such as homeostasis and activation.

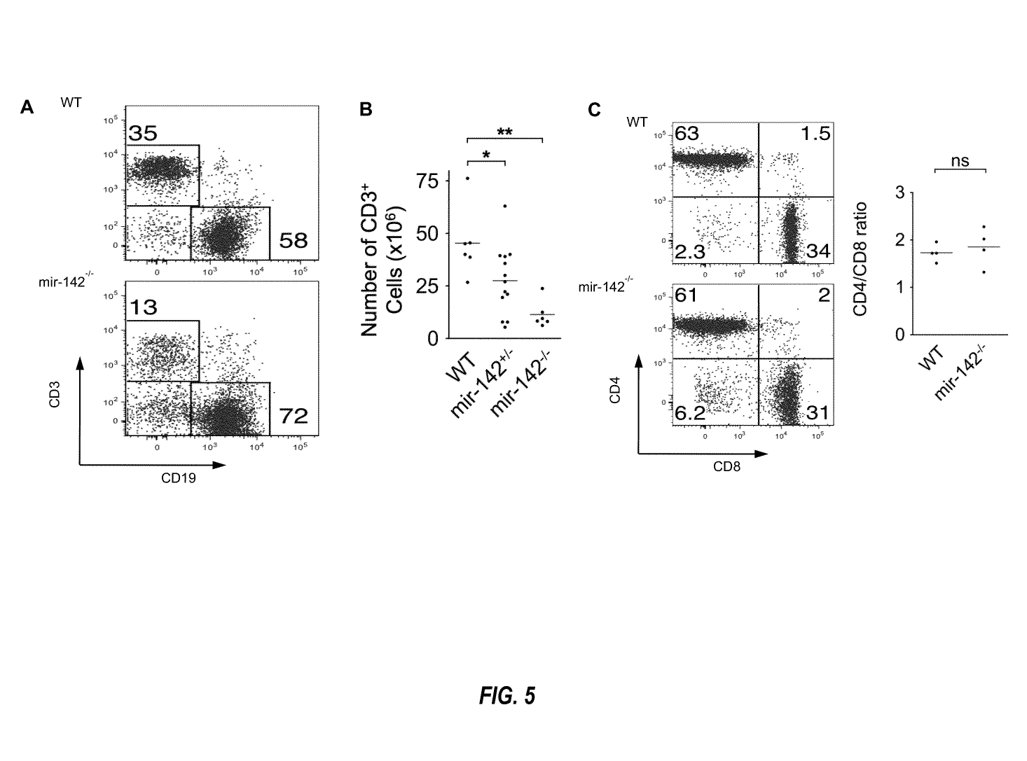

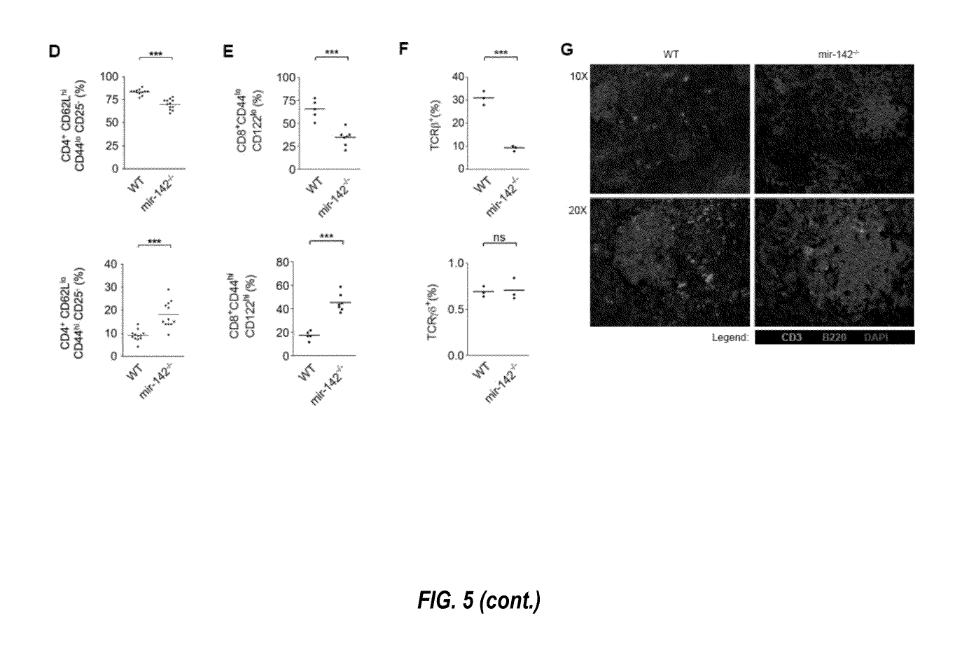

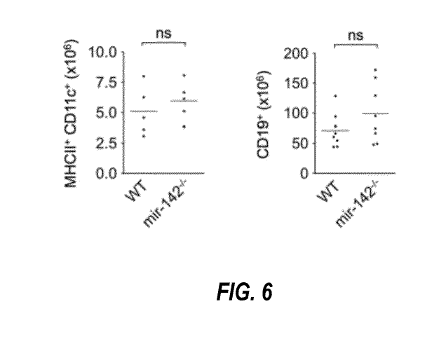

A pivotal role for mir-142 in T cell regulation was confirmed following the generation of constitutive mir-142 deficient mice (FIGS. 1-3). Offspring of heterozygous mir-142^{+/-} mice were born at the expected Mendelian ratio and homozygous mir-142^{-/-} remained healthy beyond one year of age (data not shown). Flow cytometric analysis revealed a striking reduction in the total number of CD3⁺ T cells in the absence of mir-142 when compared with mir-142⁺⁺wild-type (WT) controls (FIG. 5a-b,f) with no alteration in the ratio of CD4⁺:CD8⁺in mir-142^{-/-} compared to WT mice (FIG. 5c). Although mir-142 is expressed across multiple haematopoietic lineages⁶, mir-142 deficiency resulted in an exclusive reduction in the cellularity of the T cell compartment, as the numbers of other splenic and lymph node lineages such as B cells and dendritic cells were not significantly different in mir-142^{-/-} mice (FIG. 5d-e, and FIG. 6). These data, however, do not exclude other defects in these and other lineages of immune cells in mir-142^{-/-} mice. Analysis of mir-142^{+/-} mice showed an intermediate homeostatic naïve CD4⁺ T cell defect (FIG. 5b) associated with expression levels at 50% compared with WT mice (FIG. 2c), indicative of a gene dose effect for this phenotype.

Previous studies have shown a relative dominance of memory T cells in lymphopenic mice⁷. In agreement with this, T cell lymphopenia in mir-142^{-/-} mice resulted in an increased percentage of CD4⁺memory (CD4⁺CD25⁻CD62L^{low} CD44^{high}, 9.1% (WT) vs. 18% (mir-142^{-/-}) cells in comparison to naïve (CD4⁺CD25⁻CD62L^{high} CD44^{low}, 85% (WT) vs. 70% (mir-142^{-/-}) cells) (FIG. 5d). The percentage of memory CD8⁺ T cells was also increased in mir-142^{-/-} mice (CD8⁺CD44^{high}CD122^{high}, 17.6% (WT) vs. 45.6% (mir-142^{-/-})) and naïve CD8⁺ T cells reduced (CD8⁺CD44^{low}CD122^{low}, 65.6% (WT) vs. 34.9% (mir-142^{-/-})) (FIG. 5e).

Immunohistochemistry demonstrated a marked reduction of CD3⁺ T cells in the mir-142^{-/-} spleen when compared with WT, although splenic structural organisation appeared grossly normal with follicular development evident (FIG. 5f). Thus mir-142 is essential for the maintenance of T cell numbers and the absence of mir-142 results in a profound homeostatic defect in the T cell lineage.

### 5.3 Example 3

### The Homeostatic Defect in Naïve CD4⁺ T Cells from mir-142^{-/-} Mice is Post-Developmental

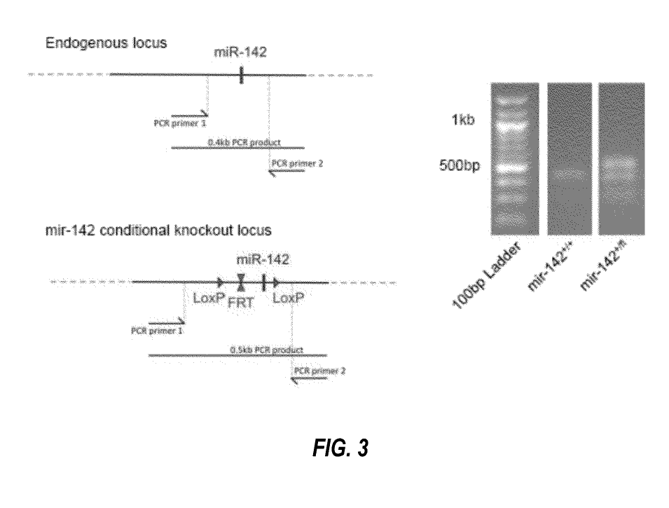

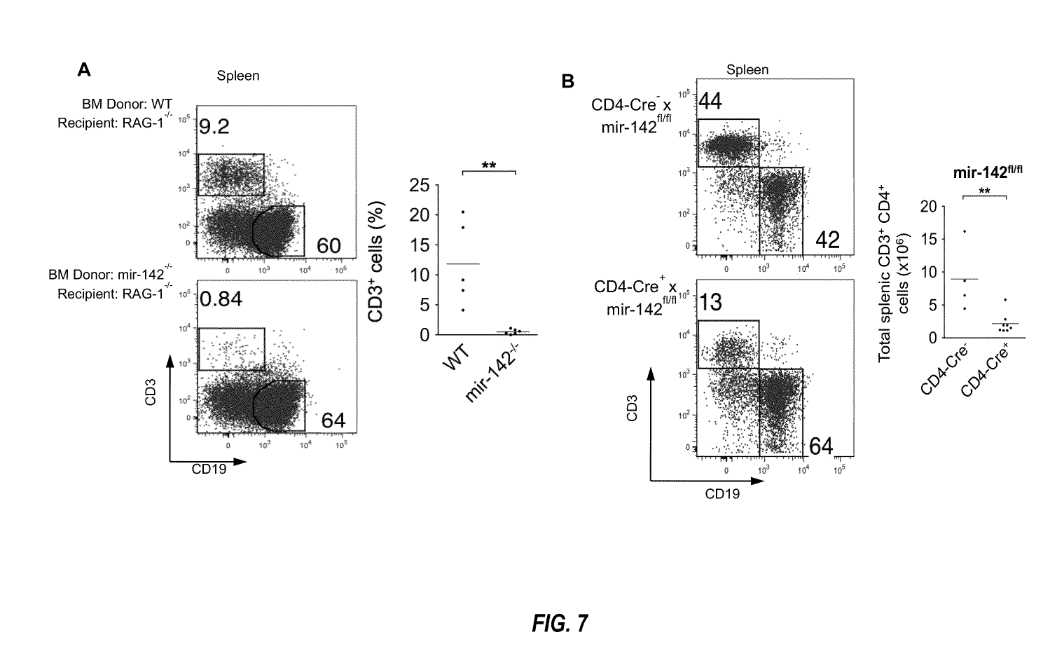

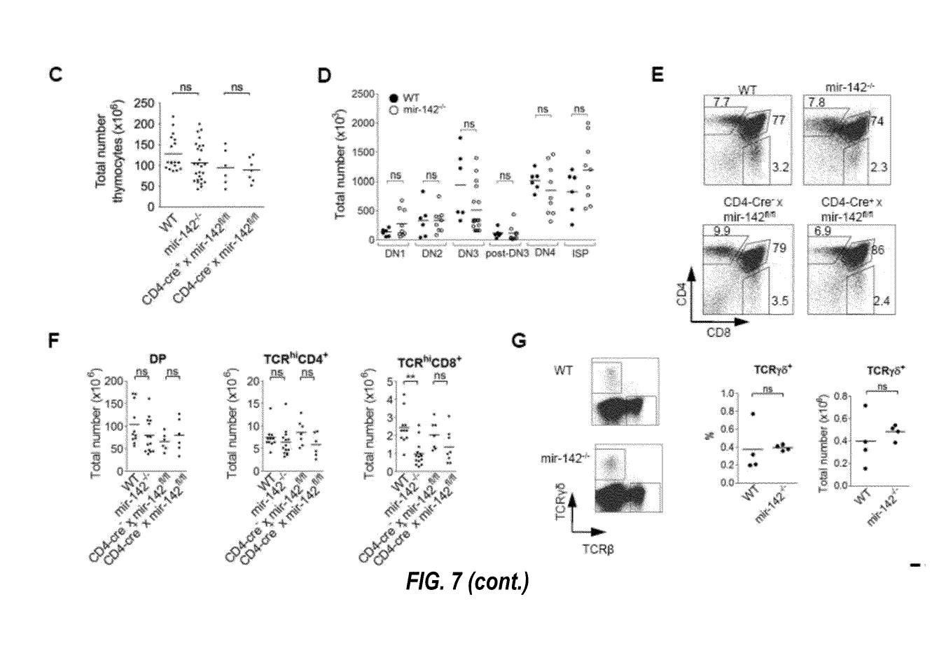

In order to address whether this T cell homeostatic defect resulted from lymphoid or non-lymphoid deficiency of mir-142 we generated bone marrow chimeras by reconstitution of sublethally-irradiated RAG1^{-/-}recipient mice with bone marrow from either mir-142^{-/-} or WT donors. At 4 weeks post-transfer, CD3⁺ T cells were virtually absent in the spleen and lymph nodes of recipients of mir-142^{-/-} bone marrow, despite CD19⁺ B cells having reconstituted equally in recipients of both mir-142^{-/-} and WT cells (FIG. 7a). To confirm the tissue specificity of this effect, we generated mice with a conditional allele of mir-142 (mir-142^{fl/fl} mice, FIG. 3). These mice were then crossed with CD4-Cre mice in order to examine the CD4⁺ lineage specificity of this phenotype. Conditional deletion of mir-142 in cells expressing CD4 recapitulated the defect observed in constitutive mir-142^{-/-} mice, demonstrating that the homeostatic defect is T cell lineage-specific (FIG. 7b).

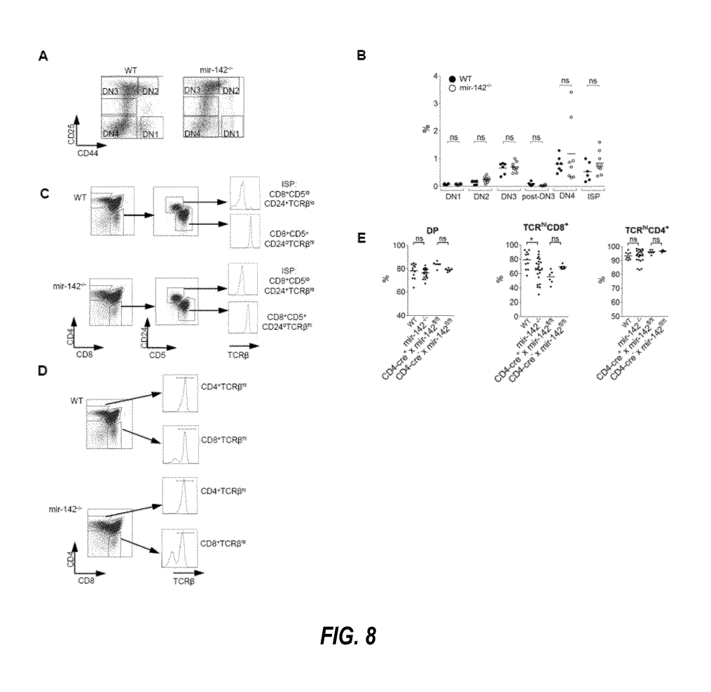

Mir-142^{-/-} and CD4-cre⁺x mir-142^{fl/fl} mice did not display significantly reduced thymocyte cellularity (FIG. 7c). Analysis of early thymic populations (DN1-DN4 and immature CD8⁺(ISP)) and gamma delta TCR⁺ thymocytes revealed no significant changes in mir-142^{-/-} mice (FIG. 7d and FIG. 8b,f). The absence of mir-142 resulted in a significant reduction in mature thymic TCR^{hi}CD8⁺ T cells, but not mature TCR^{hi}CD4⁺ T cells or double positive (DP) thymocytes (FIG. 7e-f and FIG. 8e). Interestingly, the reduction of TCR^{hi}CD8⁺ T cells in mir-142^{-/-} mice was partially corrected when mir-142 was deleted at a later stage of development in CD4-cre⁺x mir-142^{fl/fl} mice, while TCR^{hi}CD4⁺ T cells and DP thymocytes remained comparable to those in WT mice (FIG. 7f and FIG. 8e). Analysis of proliferating thymocytes by BrdU incorporation demonstrated a tendency towards reduced proliferation in mir-142^{-/-} thymocytes (FIG. 7g). This indicates that the absence of mir-142 results in a defect in CD8⁺ thymocytes, while thymic CD4⁺ T cell development remains largely unaffected at this level of analysis.

### 5.4 Example 4

### Mir-142 Regulates CD4⁺ T Cell Lymphopenia-Induced Proliferation in a Cell-Intrinsic Manner

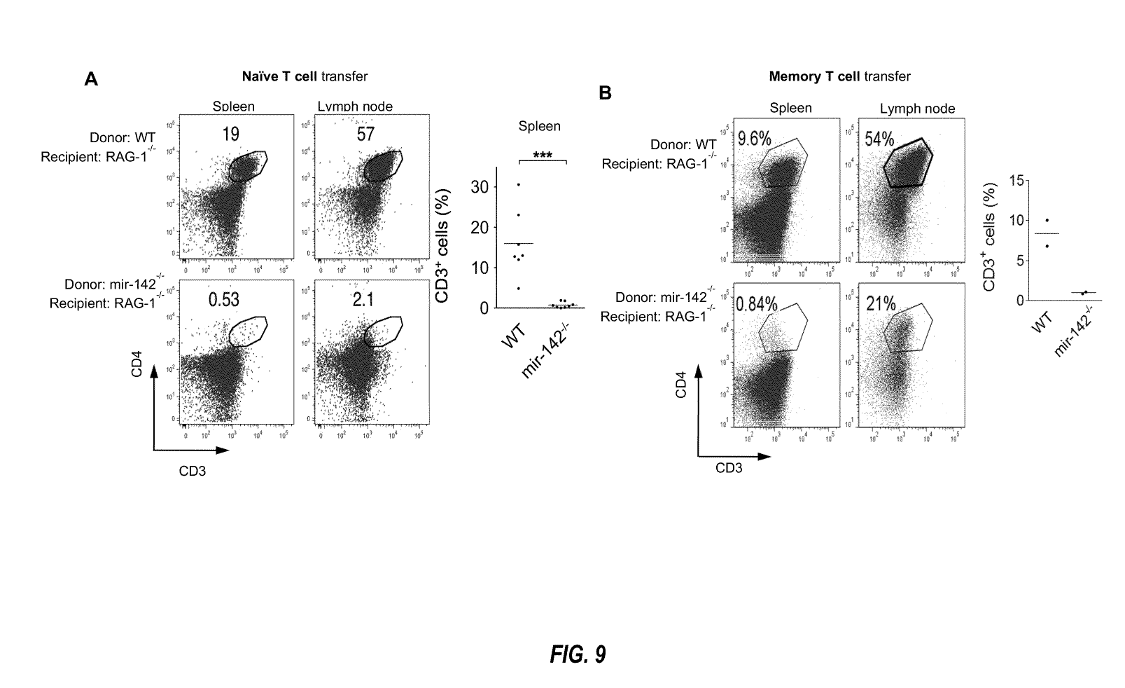

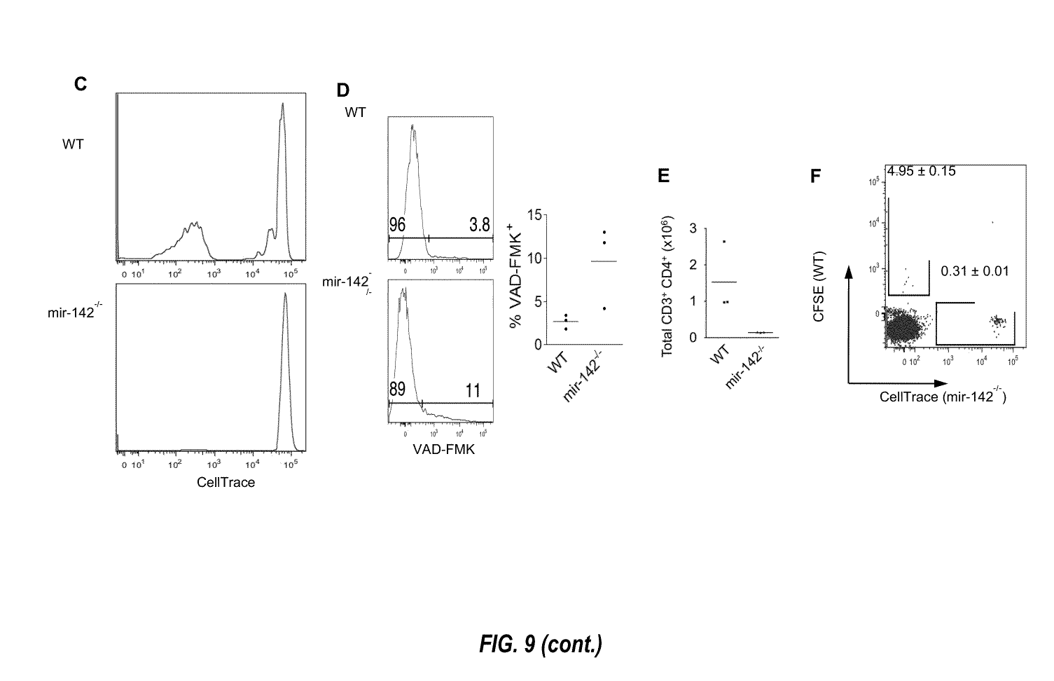

Given that thymic development of CD4⁺ T cells was normal in mir-142^{-/-} mice, we focused on peripheral CD4⁺ T cell homeostasis. Transfer of naïve CD4⁺ T cells into RAG-1^{-/-} mice results in lymphopenia-induced proliferation (LIP), a process that is dependent on similar immunological cues as steady-state homeostasis'. In order to investigate the mechanisms responsible for the peripheral homeostatic defect of mir-142^{-/-} naïve CD4⁺ T cells, we transferred WT or mir-142^{-/-} CD4⁺ CD62L^{high} CD44^{low}CD25⁻ naïve T cells (Thp) into RAG-1^{-/-} mice. At 4 weeks post-transfer, T cells were observed in the spleen and lymph nodes of mice that had received WT cells, but were essentially absent in the recipients of mir-142^{-/-} T cells (FIG. 9a). Furthermore, mir-142^{-/-} T cells were not detected in any other organs such as lung and liver, indicating that this was not a primary defect in T cell trafficking to lymphoid tissue (data not shown). Transfer of CD4⁺ memory T cells also demonstrated impaired reconstitution of recipient mice by mir-142^{-/-} cells in the spleen, although a relatively higher percentage of cells were detected in peripheral lymph nodes. However, this was still reduced in comparison to recipients of WT CD4⁺ memory T cells (FIG. 9b). This finding is consistent with the higher proportion of CD4⁺ memory cells previously observed in the mir-142^{-/-} mouse and may reflect a differential reliance on survival factors between naïve and memory CD4⁺ T cells⁸.

The failure of mir-142^{-/-} T cells to undergo LIP could be due to lack of response to in vivo signals that result in reduced proliferation. To address this, we tracked adoptively transferred CFSE labelled T cells and found that mir-142^{-/-} Thp had failed to divide following transfer, in contrast to WT cells where T cell proliferation had been initiated at the same time point (FIG. 9c). Furthermore, mir-142^{-/-} cells displayed higher levels of apoptosis than WT in vivo, with all (n=33) recipients of mir-142^{-/-} Thp displaying higher numbers of (CD3⁺CD4⁺) VAD-FMK⁺ cells (FIG. 9d). Thus, as a result of both impaired proliferation and increased apoptosis, the number of mir-142^{-/-} T cells was dramatically reduced when compared with WT (FIG. 9e). Failure to respond to in vivo survival signals was not restored by co-transfer of equal numbers of WT naïve T cells. Co-transferred WT cells survived and underwent robust proliferation. In marked contrast, mir-142^{-/-} cells failed to proliferate and displayed a comparative 16-fold reduction in T cell numbers (FIG. 9f). Collectively, these data show that mir-142 controls naïve T cell homeostasis in vivo in a T cell-intrinsic manner.

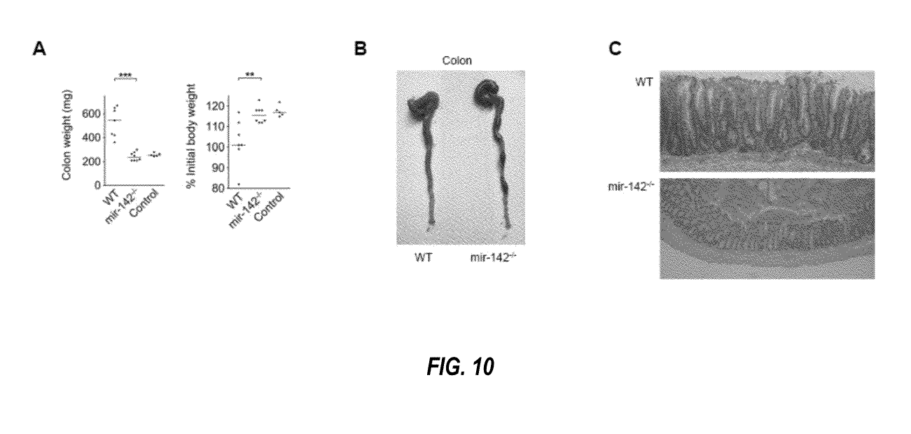

Dysregulation of homeostasis of naïve CD4⁺ T cells has been implicated in a number of pathological clinical outcomes, including inhibited tumour clearance and propagation of chronic inflammatory bowel disease⁹. Modulation of mir-142 in these settings could therefore represent a novel potential treatment. To examine this possibility, we used a naïve T cell transfer model of colitis¹⁰ (FIG. 10). Thp from WT and mir-142^{-/-} mice were transferred into RAG-1^{-/-} mice. Recipients of WT cells developed fulminant colitis characterized by weight loss, increased colon weight and characteristic remodelling of the colonic mucosa (FIG. 10a-c). However, mice that received Thp from mir-142^{-/-} mice were completely protected from disease induction as indicated by body and colon weight comparable to control non-colitic mice.

### 5.5 Example 5

### CD4+ T Cell Activation is Unimpaired in the Absence of mir-142

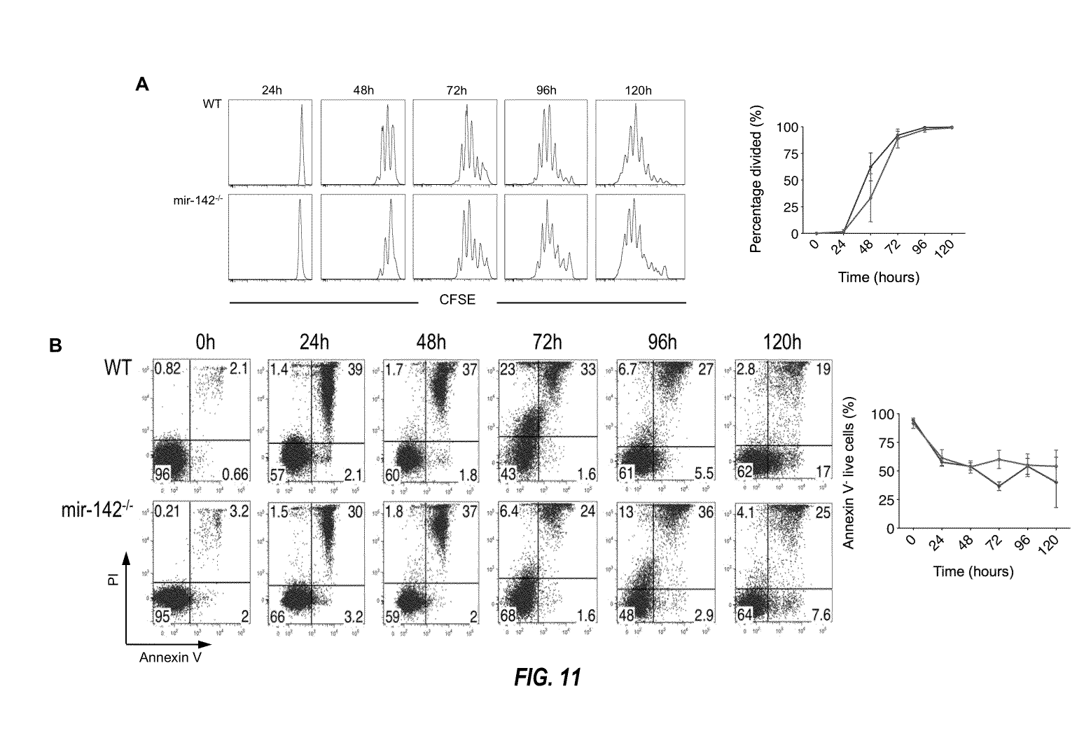

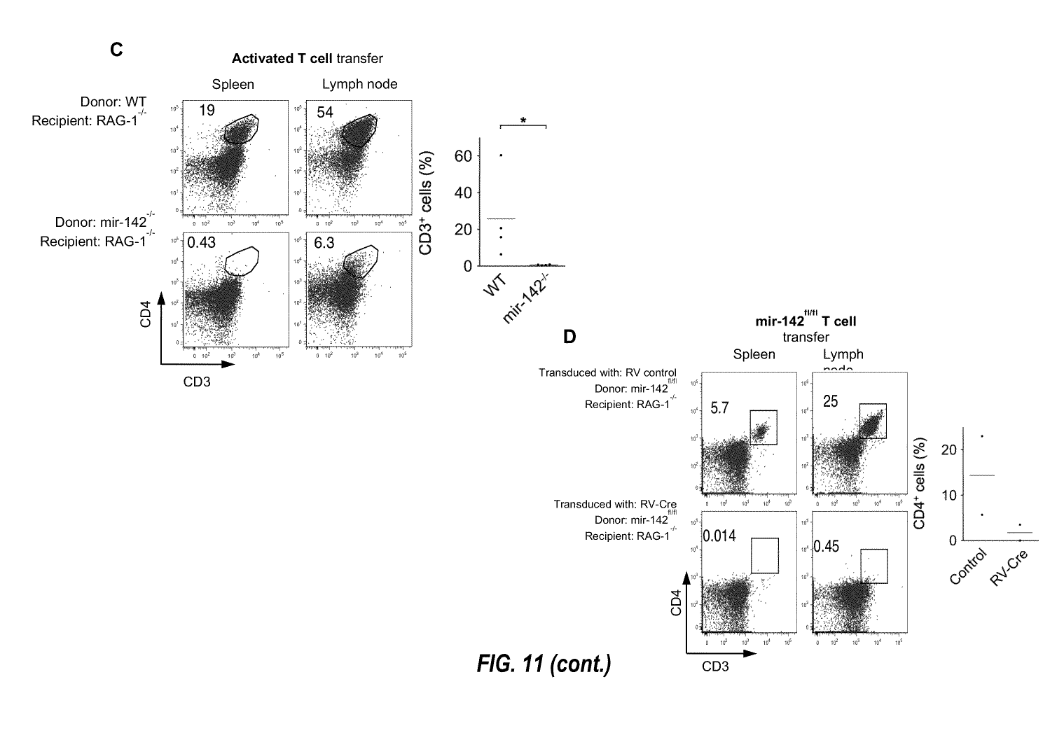

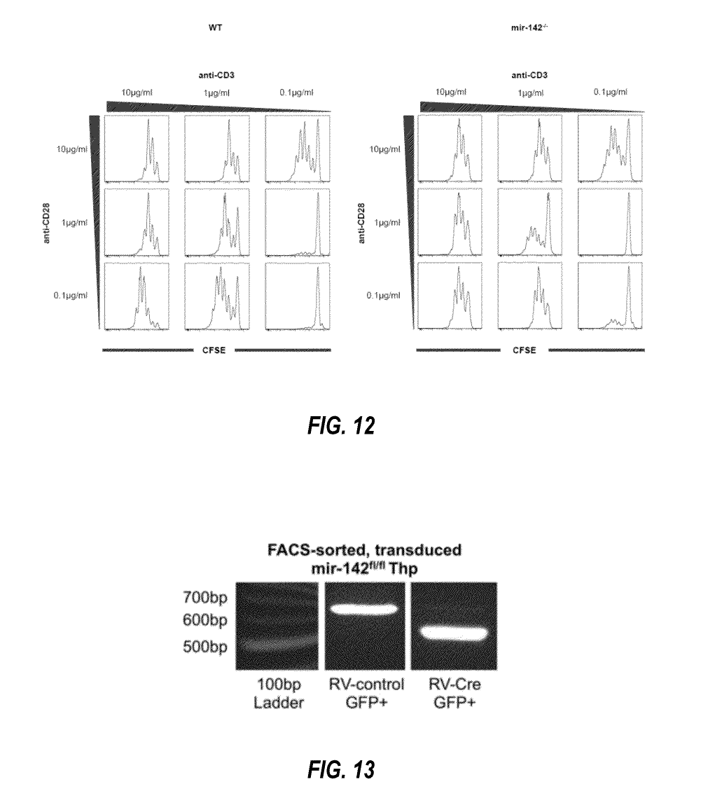

Both homeostatic and lymphopenia-induced proliferation require TCR signalling. To assess if the inability of mir-142^{-/-} CD4⁺ T cells to expand in the same way as WT cells after in vivo transfer results from a defect in TCR signalling we examined the proliferation of mir-142^{-/-} T cells during in vitro TCR stimulation. CFSE dilution demonstrated that proliferation was comparable between WT and mir-142^{-/-} T cells activated in vitro (FIG. 11a), even when the activation stimuli were titrated to lower levels or when antigen presenting cells were used (FIGS. 10 and 12). Interestingly, no difference in apoptosis was observed between WT and mir-142^{-/-} TCR activated T cells (FIG. 11b). Having shown that the proliferation and survival of activated naive T cells was unaffected in vitro, we investigated whether homeostasis in vivo was affected after optimal in vitro activation. Transfer of WT and mir-142^{-/-} T cells activated for 7 days in vitro into RAG-1^{-/-} mice showed that, despite TCR activation, long-term survival of these mir-142^{-/-} T cells remained defective to a similar extent to naive mir-142^{-/-} CD4⁺ T cells in vivo (FIG. 11c).

To delineate further the stage at which mir-142 deficiency causes these abnormalities, mir-142 was deleted in vitro at 24 hours after activation, by transduction of mir-142^{fl/fl} Thp with Cre-expressing retrovirus (FIG. 13). Transduced cells were then cultured for one week in vitro, purified by sorting GFP⁺ cells and transferred into RAG-1-mice. In vitro cell survival was unimpaired (data not shown), however mir-142 deficient cells recovered at 3 weeks following transfer were reduced (n=22) when compared with control-transduced cells (FIG. 11d). This shows that mir-142 insufficiency in mature CD4⁺ T cells results in marked abnormalities of T cell homeostasis in vivo. These defects are not apparent during in vitro activation and culture, even at suboptimal levels of costimulation or TCR ligation.

### 5.6 Example 6

### IL-7 Responsiveness is Impaired in mir-142 Deficient CD4⁺ T Cells

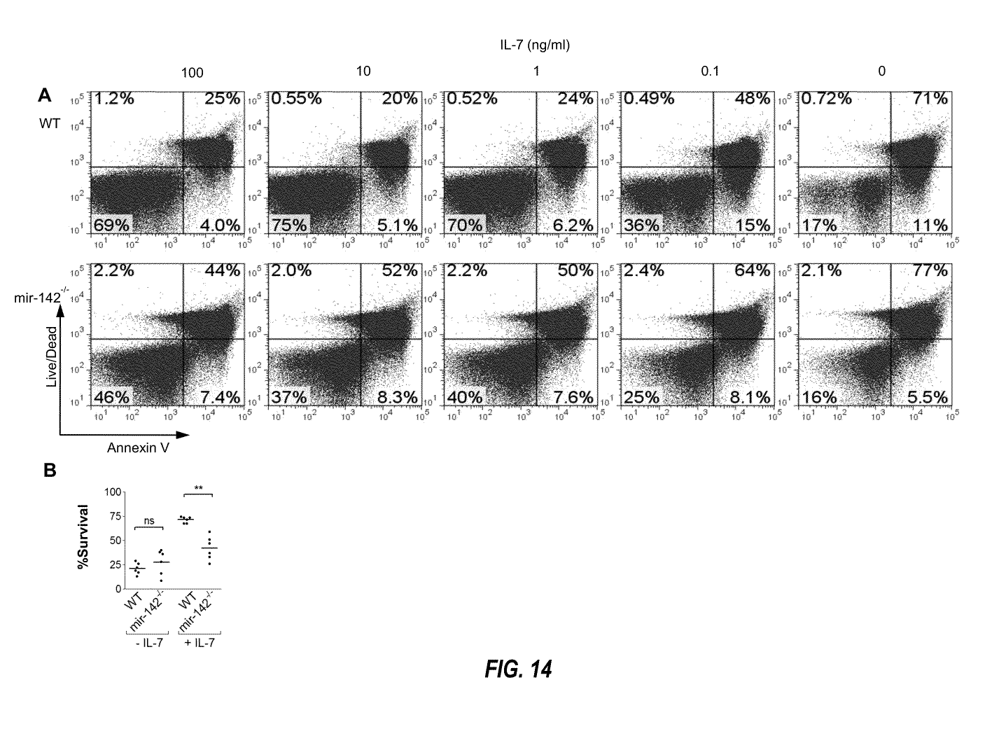

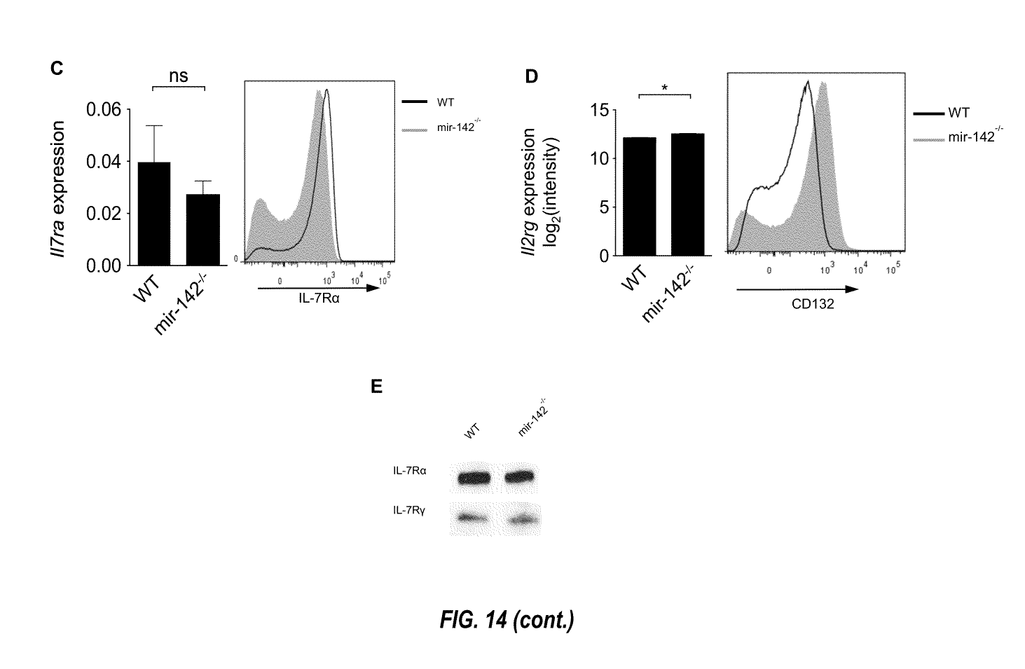

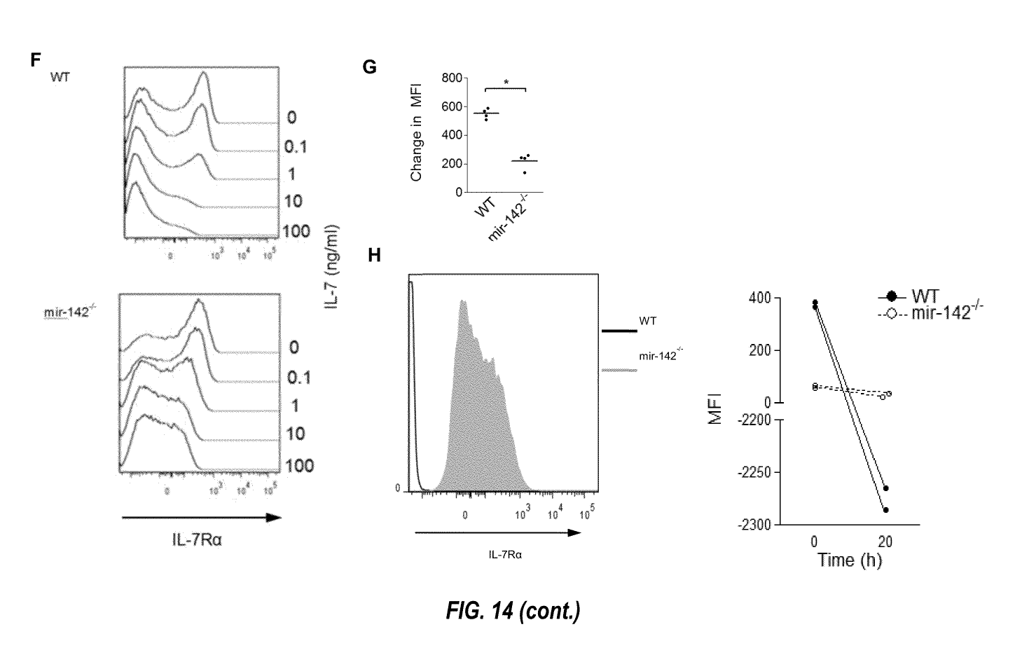

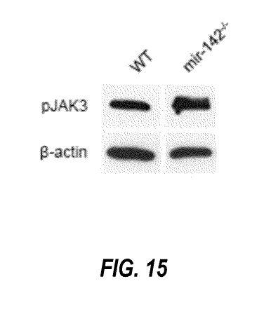

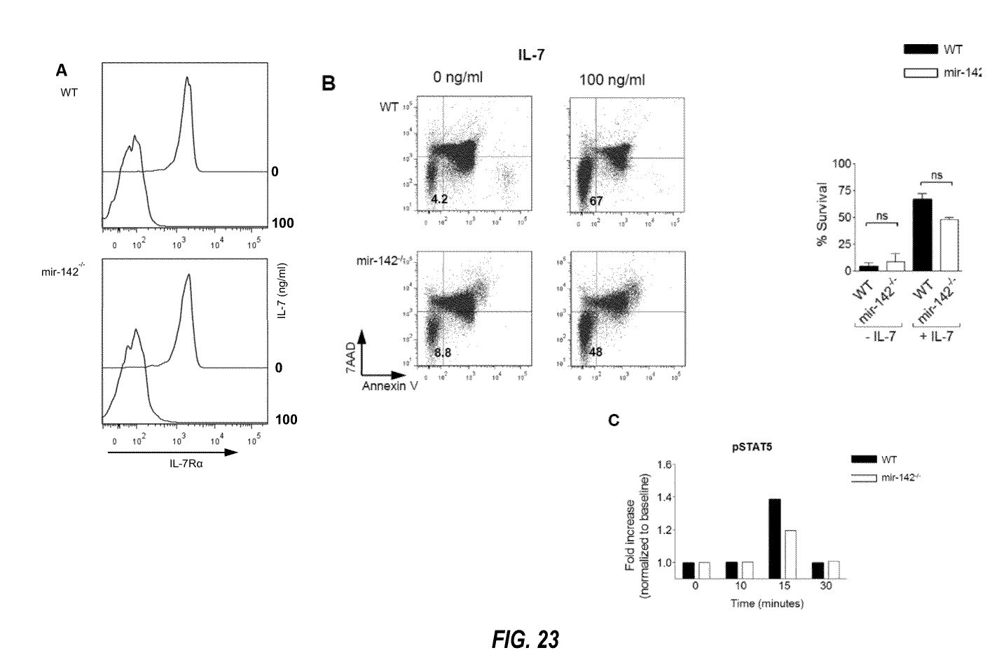

In addition to TCR signalling, naïve CD4⁺ T cell homeostasis is critically dependent on survival signals downstream of the IL-7 receptor (IL-7R). Given that TCR responses of mir-142^{-/-} cells were intact, we sought to determine whether mir-142 deficient T cells were able to respond to IL-7. We first tested whether Thp were able to respond to recombinant exogenous IL-7 in vitro. The survival of WT Thp was supported by IL-7 whereas mir-142 Thp survived poorly at all doses of IL-7, even when supraphysiological levels were used (FIGS. 14a-b). Directly ex vivo, we observed that surface expression of IL-7Rα was slightly reduced in mir-142^{-/-} Thp, although mRNA levels were not significantly different from WT (FIG. 14c), whilst expression of the common gamma chain (I12rg) was increased in mir-142^{-/-} Thp at both the cell surface and mRNA levels (FIG. 14d). Ligation of IL-7 followed by immunoprecipitation of the IL7Ra demonstrated equal amounts of coprecipitated IL-7Rγ in WT and mir142^{-/-} T cells, excluding IL-7Rγ sequestration by other cytokine receptors (FIG. 14e). In addition levels of phosphorylated JAK3 were unchanged (FIG. 15), demonstrating that receptor subunit expression and function are not deficient in the absence of mir-142. Following IL-7R ligation, expression of the receptor complex is down-regulated in WT T cells (FIG. 14f-g). Despite intact resting receptor expression, mir-142^{-/-} CD4⁺ T cells failed to down-regulate IL-7Rα expression to the same level as WT T cells, in response to IL-7 in vitro (FIG. 14f-g). IL-7-mediated survival of peripheral naive CD8⁺ T cells from mir-142^{-/-} mice was reduced in mir-142^{-/-} (FIG. 25), as was pSTAT5 in response to IL-7 (FIG. 25b), indicating that defective thymic IL-7R signalling is maintained in peripheral naïve CD8⁺ T cells.

In order to assess the effect of IL-7 signalling in vivo, we compared IL-7Rα downregulation in WT and mir-142^{-/-} CD4⁺ T cells in the same host. This was necessary because comparing IL-7R expression in the lymphopenic mir-142^{-/-} mouse to a lymphocyte-sufficient WT mouse, is not representative of the response of mir-142^{-/-} CD4⁺ T cells to IL-7 in vivo, as IL-7 levels are regulated by consumption¹¹. In order to normalise endogenous IL-7 levels, we co-transferred equal numbers of naive T cells from WT and mir-142^{-/-} mice, labelled with CFSE and CellTrace Violet respectively into RAG1^{-/-} hosts and analysed IL-7Rα expression 20 h later¹². We found that IL-7Rα expression levels following transfer into RAG1^{-/-} mice were dramatically downregulated in WT Thp, whereas expression remained unchanged in mir-142^{-/-} Thp (FIG. 14h). Failure to downregulate IL-7Rα demonstrates that IL-7 signalling in mir-142^{-/-} Thp is defective in vivo. Thus, mir-142 is critically required for responsiveness to IL-7 survival signals in naïve CD4⁺ T cells both in vitro and in vivo.

### 5.7 Example 7

### IL-7 Induced STAT5 Phosphorylation is Defective in mir-142^{-/-} T Cells

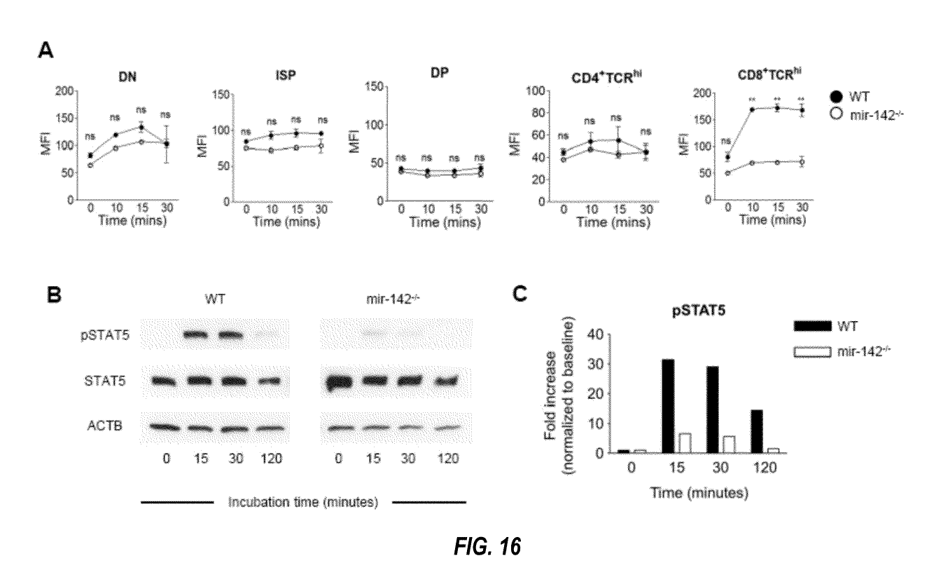

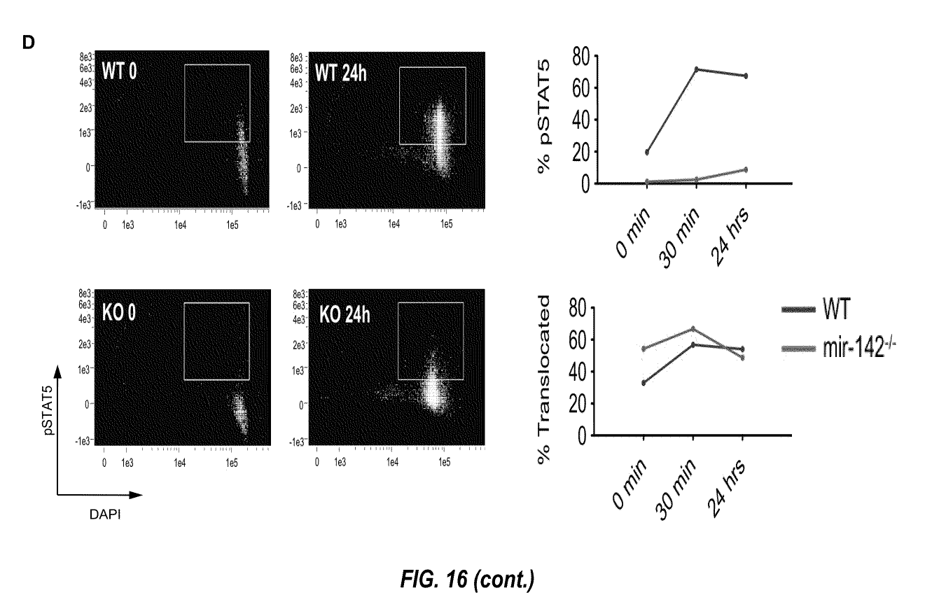

Phosphorylation of STAT5 is a proximal consequence of IL-7R ligation (reviewed in ¹³). IL-7R signalling is also critical for normal T cell development in the thymus. In the absence of mir-142, phosphorylation of STAT5 in response to IL-7 stimulation was unaffected in early thymocytes (DN and ISP) (FIG. 16a). Consistent with the low level of IL-7R expression on DP thymocytes, phosphorylation of pSTAT5 was not detected. However, pSTAT5 levels in CD8⁺TCR^{hi}, but not CD4⁺TCR^{hi}, mir-142^{-/-} thymocytes were significantly reduced (FIG. 16a). This defect in phosphorylation of STAT5 in response to IL-7 in mir-142^{-/-} CD8⁺ T cells was maintained in peripheral naïve CD8⁺ T cells (FIG. 16b). Similarly, STAT5 was phosphorylated rapidly in peripheral WT CD4⁺ T cells, however, pSTAT5 levels were markedly reduced in mir-142^{-/-} T cells in response to IL-7 (FIGS. 16c-e). In addition, STAT5 phosphorylation was not maintained in mir-142^{-/-} T cells, as pSTAT5 was not detectable in mir-142^{-/-} CD4⁺ T cells 2 h post IL-7 stimulation, whereas low levels could still be detected in WT CD4⁺ T cells (FIG. 16c). Flow cytometric analysis confirmed that STAT5 phosphorylation was reduced from over 71% in WT T cells to 8.7% in mir-142^{-/-} T cells after 30 minutes of culture in the presence of IL-7 (FIG. 16e). However, in the small number of mir-142^{-/-} T cells that had phosphorylated STAT5, comparable levels of nuclear translocation were observed when compared with WT mice (FIG. 16e). This indicates that proximal IL-7R signalling is inhibited at the level of STAT5 phosphorylation in the absence of mir-142. These data show that the phenotype of markedly disordered T cell homeostasis in the absence of mir-142 is likely due to impaired IL-7R signalling caused by failure to phosphorylate STAT5. These data demonstrate that the impaired CD4⁺ T cell homeostasis in the absence of mir-142 is due to impaired IL-7R signalling caused by failure to phosphorylate STAT5 in vivo.

### 5.8 Example 8

### Discussion

MicroRNAs have emerged as powerful regulators of a wide range of biological processes, including cellular homeostasis. The ability of homeostatic mechanisms to regulate the naïve T cell compartment is fundamental for the maintenance of peripheral T cell numbers. Here we report that mir-142 is amongst the most abundantly expressed microRNA in naïve T cells, and identify mir-142 as a critical regulator of T cell development and homeostasis. The absence of mir-142 results in aberrant CD8⁺ development in the thymus, with CD8⁺ T cells unable to signal via the IL-7R. In addition, the absence of mir-142 results in a profound survival defect in naïve T cells in vivo, as evidenced by greatly reduced peripheral T cell numbers. Early apoptosis of naïve CD4⁺ T cells in mir-142^{-/-} mice was due to inhibition of IL-7R signalling.

Thymic selection of T cells is critical for the generation of functional non-autoreactive T cells for replenishment of the peripheral T cell pool. Although we found significantly decreased numbers of both CD4⁺ and CD8⁺ lineages in the periphery of mir-142^{-/-} mice, our data supports a larger role of mir-142 in the thymic development of the CD8⁺ lineage, rather than the CD4⁺ lineage. Mature CD8⁺TCR^{hi} T cells were reduced and proliferated to a lesser degree in mir-142^{-/-} mice, whereas CD4⁺ SP thymocytes were relatively unaffected. Thymic TCR^{hi}CD4⁺ T cell development was comparable to WT mice even when mir-142 was silenced at a later stage (DP) of development in CD4-cre⁺ x mir-142^{fl/fl} mice, suggesting that the CD4⁺ homeostatic defect in mir-142^{-/-} mice is largely peripherally mediated, while CD8⁺ T cells require mir-142 expression for IL-7 signalling and normal thymic development. However, we cannot formally exclude more subtle defects of CD4⁺ T cell development.

T cell homeostasis is essential to normal function of the immune system. It is known that TCR/MHC interactions and IL-7 are non-redundant for this process¹. IL-7 signalling contributes to T cell survival via the increased expression of the anti-apoptotic molecule B-cell leukemia/lymphoma 2 (Bcl-2)¹⁹. Drastically dysregulated homeostasis of the CD4⁺ T cell niche in the absence of mir-142 in vivo can be in large part attributed to selective inhibition of IL-7 responsiveness, without affecting TCR signalling.

IL-7 is known to activate multiple signalling pathways including the PI3K/AKT pathway and the STAT5 pathway. IL-7R ligation results in IL-7R down-regulation, a process controlled at a number of molecular levels. Following ligation, IL-7R is internalised resulting in down-regulation of surface expression. This down-regulation reduces competition for remaining cytokine in an IL-7-limited environment. In addition, IL-7R ligation modulates its own expression at a transcriptional level in a complex negative feedback loop (reviewed in ²⁰). Despite the absolute requirement for IL-7R signalling for survival of both subsets of T cells, a study by Park et al. demonstrated that cytokine-mediated transcriptional down-regulation of IL-7Rα was controlled by different molecular mechanisms in CD4⁺ versus CD8⁺ T cells²¹. In CD8⁺ T cells, IL-7 induced down-regulation of IL7Rα was mediated by GFI1. However, GFI1 was not involved in IL-7Rα down-regulation in CD4⁺ T cells. The data presented here suggest that mir-142 is a selective regulator of IL-7Rα signalling in CD4⁺ T cells.

The augmented development of memory CD4⁺ T cells during lymphopenia is well documented, although the mechanisms are not fully understood²². The memory cell pool consists of antigen-specific T cells, reactive to either foreign or self-antigens and those that arise from antigen-independent expansion of naive T cells during lymphopenia-induced proliferation. CD4⁺ memory T cells are also dependent on IL-7. However, the ability of IL-15 to sustain antigen specific CD4⁺ memory responses has also been reported⁸. The relative abundance of memory CD4⁺ T cells in mir-142^{-/-} mice may reflect a certain level of redundancy of gamma chain cytokines in the ability to support survival of antigen-specific memory cells.

The finding that mir-142 dictates IL-7 responsiveness in CD4⁺ T cells reveals a previously-unknown mechanistic pathway controlling T cell survival. Our results provide new insights into the regulation of this process. Alterations in the IL-7 response have important pathological implications and abnormalities in this pathway have been identified in a number of autoimmune disorders, inflammatory diseases and immunodeficiencies. For example, increased levels of IL-7 have been found in the inflamed mucosa of patients with Crohn's disease, and there is strong evidence to suggest that excess IL-7 in these patients results in increased proliferation and production of IFN-γ by mucosal T cells, thus contributing to pathology³⁰. Additionally, genetic polymorphisms in the IL-7R are associated with human autoimmune conditions such as multiple sclerosis and inflammatory bowel disease³¹⁻³⁴. In contrast, impaired IL-7R signalling has been observed in HIV-infected CD4⁺ T cells although the mechanisms are not well understood³⁵. The potential of IL-7 administration for immune reconstitution in conditions where T cell homeostasis is disturbed has aroused intense interest and a number of clinical studies have already been performed³⁶.

In summary, mir-142 is a novel regulator of IL-7 responsiveness in naïve CD4⁺ T cells that controls the homeostatic maintenance of the T cell niche. These findings are of profound importance for the understanding of naïve CD4⁺ T cell responsiveness to survival cues and identify mir-142 as a target for modulation of IL-7 responsiveness in HIV, cancer and autoimmunity.

### 5.9 Example 9

### Materials and Methods for Example 10

Chromatin Immunoprecipitation.

Naïve human CD4⁺ T-cells were isolated and polarised as described (Jenner et al., 2009). Cells were crosslinked with formaldehyde, lysed and sonicated at 24 W for 10×30 second pulses using a Misonix Sonicator 3000. The resulting whole cell extract was incubated overnight at 4° C. with Dynal Protein G beads preincubated with 10 1 of purified serum (T-bet antibody 9856⁵¹ or 10 g of anti-GATA3 (D-16, Santa Cruz). Beads were washed and bound complexes eluted and crosslinks reversed by heating at 65° C. IP and input DNA were then purified by treatment with RNAseA, proteinase K and phenol:chloroform extraction. ChIP-chip experiments were performed as described previously (Jenner et al., 2009). ChIP-seq libraries were constructed from IP and input DNA by standard Illumina protocols, except that DNA in the range 150-350 bp was gel-purified after PCR-amplification. The library was quantified using an Agilent bioanalzer and subjected to 35 bp single-end read sequencing with an Illumina Genome Analyzer II.

ChIP-Seq Data Analysis.

Initial processing was performed with the CASAVA pipeline. Reads were aligned to the hg18 build of the human genome with ELAND and background corrected normalized tag density calculated. Significant peaks of T-bet and GATA3 binding were identified with MACS (Zhang et al., 2008). We generally used a p-value threshold of 10⁻⁶ but varied this to identify specific combinations of T-bet and GATA3 binding. Distal binding sites (>4 kb from TSS) were assigned to the gene with the nearest TSS that was also bound proximally by T-bet or GATA3 (<2 kb from TSS). The significance of the association between transcription factor binding and change in gene expression was calculated using the hypergeometric distribution. Significant motifs were identified using MEME. Significantly enriched functional gene categories were identified using DAVID (http://david.abcc.ncifcrf.gov/).

Generation of mir-142 Deficient Mice.

Mice were generated by homologous recombination in 129Sv mouse embryonic stem cells using a targeted vector conferring neomycin resistance. This vector contained both loxP and FRT sites flanking the mir-142 locus and neomycin-resistance cassette in such a way that both conditional and constitutive mir-142 deficient mice could be generated (FIG. 1). Chimeric offspring were then bred with C57BL/6J-Cre deleter mouse to generate mice carrying the constitutive mir-142 knockout allele, or the C57BL/6J-Flp deleter mice to generate conditional mir-142 deficient mice (FIG. 2). All experimental protocols involving rodents were reviewed and approved by our local ethics review committee and the Home Office (project code: PPL706792).

Flow Cytometry.

Staining performed according to manufacturers' protocols. Samples were acquired using BD LSR II and Fortessa flow cytometers (Becton Dickinson). Data analysed with FlowJo software (Treestar).

Naïve T Cell Isolation and In Vitro Culture.

Single cell suspension was isolated from mouse lymph node and spleen. CD4⁺ T cells were then isolated magnetically using CD4 microbeads (Miltenyi Biotec). Cells were then labelled with fluorochrome-conjugated antibodies to CD4, CD62L, CD44 and CD25 (all eBioscience). Naïve T cells were sorted using a BD FACSAria II flow cytometric cell sorter to >98% purity (Becton Dickinson). Cells were initially activated for three days with plate-bound anti-CD3 (2 μg/ml) and anti-CD28 (2 μg/ml) antibodies (Bio X Cell), and cultured for a total of seven days in 10% fetal calf serum-supplemented RPMI-1640 cell culture medium (PAA) under Th0 skewing conditions in the presence of IL-2 (20 ng/ml).

T Cell Transfer.

RAG-1 deficient mice were injected intraperitoneally with cells resuspended in sterile phosphate buffered saline (PBS). Mice were weighed prior to injection and monitored for weight loss and signs of disease onset including diarrhoea, rectal bleeding, weight loss and for signs of peritonism. For naïve T cell transfer, 0.5×10⁶ cells were injected.

CFSE Cell Division Analysis.

For CFSE (Invitrogen) tracking experiments, cells were isolated as described and then labelled according to the manufacturer's protocol with CFSE (2 μM).

Microarray.

Total RNA was extracted using Trizol (Invitrogen), then labelled and hybridized to the Affymetrix Mouse Gene ST 1.0 microarray according to the manufacturer's instructions. Data were normalized using the robust multi-array average (RMA) method as described previously (Irizarry et al., 2003).

Real-Time RT-PCR.

mRNA and miRNA expression was quantified using Taqman assays (Applied Biosystems). Reactions were performed according to the manufacturer's instructions and analysed using a 7500 real-time PCR instrument (Applied Biosystems).

miRNA Target Prediction.

The target analysis software packages StaRmiR (accessed from sfold.wadsworth.org/cgi-bin/starmir.pl) (Kertesz et al., 2007) and RNAhybrid (accessed from bibiserv.techfak.uni-bielefeld.de/rnahybrid/) (Rehmsmeier et al., 2004) were employed in order to detect potential binding sites, and both identified multiple partial sites for miR-142-3p in the T-bet 3′UTR.

Luciferase.

PsiCHECK2 vector was obtained from Promega. 3′UTR target sequence was cloned from murine T cell cDNA using the primers indicated in Supplementary Table 1. For luciferase assays, 293T cells were seeded at 1×10⁴ cells per well of a 24 w plate and maintained in DMEM+10% FCS (both supplied by PAA) for 48 hours until approximately 50% confluent. Calcium phosphate transfection was performed with 5 ng of PsiCHECK2 vector and 0.5 ug of targeting vector. Luciferase assay was performed at 24 h post-transfection using the Promega Dual Luciferase Reporter Assay system, according to the manufacturer's instructions.

### 5.10 Example 10

### T Cell Lineage Commitment and Plasticity is Regulated by microRNA-142

We sought to test the hypothesis that key transcription factors regulate T helper cell lineage commitment through binding to specific miRNA loci. Using chromatin immunoprecipitation coupled with massively-parallel sequencing (ChIP-seq) for T-bet and GATA-3 in human Th1 and Th2 cells, we identified a number of miRNAs that were bound by either one or both transcription factors (Table 1).

TABLE 1

| | | Th1 T-bet binding | | | Th1 GATA3 binding | |
|---|---|---|---|---|---|---|
| MicroRNA | Hairpin coordinates | Binding site 1 | Binding site 2 | Binding assignment | Binding site 1 | Binding site 2 |
| hsa-mir-7-1 | chr9: 65774462-66774692 | chr17: 65764937-65785144 | | Primary transcrpt | chr9: 65784226-85784429 | |
| hsa-mir-7-1 | chr1: 154556756-154556845 | | | — | chr1: 154656735-154659936 | |
| hsa-mir-7-2 | chr5: 87996426-87995513 | | | — | chr5: 87998423-87995624 | |
| hsa-mir-21 | chr17: 55273408-55273462 | chr17: 55272873-55273074 | Chr17: 55275320-55271531 | Harpin | | |
| hsa-mir-23a | chr19: 13605400-12626473 | | | — | chr19: 13006793-13806994 | |
| hsa-mir-24-2 | chr19: 13605100-46484455 | | | — | chr19: 13808793-13606994 | |
| hsa-mir-25 | chr1: 99529118-96529202 | | | — | chr7: 99636161-99636352 | |
| hsa-mir-26b | chr2: 218975612-218975659 | chr2: 218969151-218969352 | | Primary transcrpt | chr2: 218971027-218971228 | chr2: 216972678-218972876 |
| hsa-mir-26 | chr3: 159889262-189889348 | chr3: 189354982-189356183 | | Primary transcrpt | chr3: 189886938-189887134 | chr3: 189354954-189256165 |
| hsa-mir-22 | chr9: 110848329-110848399 | chr9: 110922061-110922266 | | Primary transcrpt | | |
| hsa-mir-23a | chr22: 40626893-42626362 | | | — | chr22: 40559542-40559744 | |
| hsa-mir-92b | chr1: 123431091-123431657 | chr1: 1234249692-183429893 | | Harpin | | |
| hsa-mir-95 | chr4: 8057927-8055036 | | | — | | |
| hsa-mir-138-2 | chr16: 45449930-54450014 | | | — | chr16: 56461872-55462071 | |
| hsa-mir-142 | chr17: 23763591-63763678 | chr17: 53761274-53761475 | | Harpin | chr17: 53761701-537611502 | |
| hsa-mir-142a | chr5: 159544935-159442025 | chr5: 159845223-159846424 | | Harpin | chr5: 159845221-1598456422 | |
| hsa-mir-152 | chr19: 54593853-54625937 | | | — | chr19: 54694123-54694324 | chr19: 54696196-54696397 |
| hsa-mir-151 | chr8: 141511582-141611934 | chr8: 142681498-142081499 | | Primary transcrpt | chr8: 142031545-142061746 | |
| hsa-mir-155 | chr2: 25862162-22666227 | chr21: 256012024-25659056 | chr21: 25656126-22956307 | Primary transcrpt and harpin | chr21: 25967990-156966191 | chr2: 25657600-25657801 |
| hsa-mir-161a-1 | chr1: 197094796-197094925 | chr1: 197170700-197170901 | | Primary transcrpt | chr1: 197170640-197170641 | |
| hsa-mir-166 | chr1: 71305901-71305947 | chr1: 71306680-71306321 | | Harpin | | |
| hsa-mir-195 | chr17: 1861657-1861744 | chr17: 6859362-6359553 | | Harpin | | |
| hsa-mir-200c | chr12: 5943122-5943190 | | | — | chr12: 126940676-6941077 | |
| hsa-mir-205 | chr6: 52117105-52117191 | chr6: 52117526-52117727 | | Harpin | | |
| hsa-mir-223 | chr7: 66155436-6513546 | | | — | chrX: 65155156-65158354 | |
| hsa-mir-191 | chr20: 56526675-59628763 | chr20: 6627150-568527351 | | Harpin | | |
| hsa-mir-120a | chr8: 22156412-22165501 | chr8: 221880266-22155457 | | Harpin | | |
| hsa-mir-225 | chr7: 76142219-76142317 | chrX: 78140798-76142999 | | Harpin | | |
| hsa-mir-330 | chr19: 50634091-60634185 | chr19: 20537476-50837677 | chr19: 50638275-50838476 | Primary transcrpt | | |
| hsa-mir-340 | chr3: 179374906-179371003 | | | — | chr5: 175431294-179431495 | |
| hsa-mir-342 | chr14: 99645744-91645843 | chr14: 59401304-99601406 | chr14: 99602422-99602623 | Primary transcrpt | chr14: 99602429-99602630 | |
| hsa-mir-345 | chr14: 99648946-91644265 | | | — | chr14: 99841607-99841606 | |
| hsa-mir-361 | chr15: 6042496-52042366 | | | — | chrX: 9316643-85169044 | |
| hsa-mir-497 | chr17: 66619353-5662061 | chr17: 6661903-58620104 | | Primary transcrpt and harpin | | |
| hsa-mir-548b | chr5: 119431910-119422007 | chr8: 119432294-119439493 | | Primary transcrpt | | |
| hsa-mir-544c | chr12: 63302555-63302652 | | | — | chr12: 63289737-63289368 | |
| hsa-mir-5450-1 | chr6: 124427454-124429551 | chr8: 124477959-124478190 | | Primary transcrpt | chr8: 124477984-124478198 | |
| hsa-mir-546l | chr22: 25261177-25281289 | chr22: 25716451-25315552 | | Primary transcrpt | chr22: 28279967-28290165 | chr22: 25291954-25292155 |
| hsa-mir-546n | chr7: 34946694-34945971 | chr7: 35043572-35043873 | | Primary transcrpt | | |
| hsa-mir-546p | chr6: 1300160084-100180166 | chr8: 100264506-100264708 | | Primary transcrpt | | |
| hsa-mir-530-1 | chr7: 32298934-30296031 | chr7: 32292172-10292373 | | Primary transcrpt | chr7: 30292203-30292406 | |
| hsa-mir-533 | chr1: 10019344-100512492 | | | — | | |
| hsa-mir-565 | chr3: 115410811-115618106 | chr3: 115617475-113817676 | | Harpin | | |
| hsa-mir-569 | chr3: 172307146-172307242 | | | — | chr3: 172658723-172656924 | |
| hsa-mir-571 | chr4: 333945-334041 | | | — | chr4: 327407-321608 | |
| hsa-mir-575 | chr4: 63693513-63893607 | chr4: 82928465-83939167 | | Primary transcrpt | | |
| hsa-mir-581 | chr5: 52283090-83283166 | | | — | chr6: 53642112-63642019 | |
| hsa-mir-583 | chr2: 95440597-95440672 | chr5: 95440594-95440795 | | Harpin | | |
| hsa-mir-582 | chr5: 168623182-16062326 | | | — | chr6: 168625622-16842823 | |
| hsa-mir-590 | chr7: 73243463-73243540 | chr7: 73226492-73226693 | | Primary transcrpt | chr7: 73226439-73226640 | |
| hsa-mir-591 | chr7: 95666909-95487004 | chr7: 95787896-95788100 | chr7: 95768211-95788512 | Primary transcrpt | | |
| hsa-mir-596 | chr8: 1728603-1752860 | | | — | chr6: 1752967-1753158 | |
| hsa-mir-595 | chr8: 10930125-10930222 | chr8: 11095710-11295911 | | Primary transcrpt | | |
| hsa-mir-503 | chr10: 24604619-24604716 | chr10: 24239052-24539253 | | Primary transcrpt | | |
| hsa-mir-513 | chr12: 12505649-12626944 | chr12: 12769916-12770119 | | Primary transcrpt | chr12: 12769950-12770131 | |
| hsa-mir-517 | chr12: 10754812-10772491 | chr12: 107756276-107716576 | | Harpin | chr12: 107756344-107756541 | |
| hsa-mir-620 | chr12: 115070747-11807842 | | | — | chr12: 116198385-115195566 | |
| hsa-mir-621 | chr13: 40282901-40282997 | | | — | | |
| hsa-mir-623 | chr13: 96606355-98836483 | chr13: 98806796-98806606 | | Harpin | chr12: 98806683-92806684 | |
| hsa-mir-626 | chr15: 29771074-39771166 | chr15: 29740447-39740648 | | Primary transcrpt | | |
| hsa-mir-628 | chr15: 53452429-54432524 | | | — | chr15: 52457557-52456068 | |
| hsa-mir-629 | chr15: 66158754-62156661 | | | — | chr15: 66177295-68177495 | chr15: 65178996-68179199 |
| hsa-mir-630 | chr16: 70666611-63156161 | | | — | | |
| hsa-mir-633 | chr17: 58773507-58375401 | | | — | chr17: 58162831-58152432 | |
| hsa-mir-634 | chr17: 62212651-62213741 | | | — | chr17: 61729982-61729183 | |
| hsa-mir-639 | chr19: 14501346-14041152 | | | — | | |
| hsa-mir-640 | chr19: 19465481-19406367 | | | — | chr19: 19357416-19357419 | |
| hsa-mir-643 | chr19: 57476651-64476966 | | | — | chr19: 57484354-57484755 | |
| hsa-mir-644 | chr20: 32517790-32517464 | chr20: 22414610-32414011 | | Primary transcrpt | | |
| hsa-mir-647 | chr20: 62044427-62044523 | | | — | chr20: 62051474-62061875 | |
| hsa-mir-648 | chr22: 16843633-16843727 | | | — | | |
| hsa-mir-653 | chr22: 21495259-21749525 | chr22: 21492325-21493526 | | Harpin | chr22: 21493315-21449515 | |
| hsa-mir-652 | chrX: 109165212-10918310 | chrX: 109132270-1091132471 | | Primary transcrpt | | |
| hsa-mir-663 | chr20: 26136821-26136914 | chr20: 26138012-26138213 | | Primary transcrpt and harpin | chr22: 26136970-26137171 | chr22: 26138011-26138212 |
| hsa-mir-663b | chr2: 162731006-13227322 | chr2: 132729242-137729443 | | Harpin | chr2: 132729217-132729418 | |
| hsa-mir-671 | chr7: 150565429-150566557 | | | — | chr7: 150560363-150565389 | chr: 160561535-150161736 |
| hsa-mir-703 | chr11: 79790713-76793681 | chr11: 78830221-76320429 | | Primary transcrpt | | |
| hsa-mir-765 | chr12: 11566647-118464539 | chrX: 118666143-11666346 | chrX: 115711093-115711294 | Primary transcrpt and harpin | chrX: 11656499-115666700 | chrX: 115711535-118711251 |
| hsa-mir-874 | chr5: 137011159-137011237 | chr3: 137100906-137101107 | | Primary transcrpt | | |
| hsa-mir-877 | chr6: 30660087-30660173 | chr6: 33647106-30647306 | | Primary transcrpt | | |
| hsa-mir-865 | chr3: 10411172-10411246 | | | — | | |
| hsa-mir-937 | chr6: 144967114-144957200 | chr8: 144966629-144950830 | | Primary transcrpt and harpin | | |
| hsa-mir-938 | chr10: 29931198-297931281 | chr10: 29462352-29952553 | chr10: 2955234-29965435 | Primary transcrpt | | |
| hsa-mir-942 | chr1: 117438787-117439987 | chr1: 1174003477-117402676 | chr1: 117406195-117404399 | Primary transcrpt | chr1: 117402463-117402664 | |
| hsa-mir-943 | chr2: 1957908-1955002 | chr4: 1981574-1981775 | | Primary transcrpt | chr4: 1981565-1981766 | |
| hsa-mir-1181 | chr19: 10375133-10375214 | | | — | chr19: 10377193-100377494 | |
| hsa-mir-1200 | chr7: 34925466-169255622 | chr7: 37453794-37453995 | | Primary transcrpt | chr7: 37453767-37451968 | chr7: 37454996-37465199 |
| hsa-mir-1206 | chr5: 129042090-129342123 | | | — | chr6: 129041871-129042072 | |
| hsa-mir-1233 | chr15: 12461581-32461643 | | | — | chr16: 32616645-32516070 | chr15: 32517248-32517449 |
| hsa-mir-1244 | chr2: 232266267-232266382 | | | — | chr2: 32281128-232281329 | chr2: 232283086-232263267 |
| hsa-mir-1248 | chr3: 187871545-187987240 | chr3: 1579837393-127983994 | | Primary transcrpt | | |
| hsa-mir-1249 | chr22: 434975498-42975564 | | | — | chr22: 44015754-42015955 | |
| hsa-mir-1255a | chr4: 122470481-102470594 | chr4: 122668226-102444437 | | Harpin | chr4: 102468232-102468433 | chr4: 102488519-122485720 |
| hsa-mir-1256 | chr1: 21157293-21187512 | chr1: 21374491-21374892 | | Primary transcrpt | chr1: 21374512-21374713 | |
| hsa-mir-1270 | chr19: 20371039-20571162 | | | — | chr19: 20399604-20399605 | |
| hsa-mir-1277 | chrX: 1174043484-117404452 | | | — | | |
| hsa-mir-1278 | chr1: 191372216-191372336 | chr1: 191255147-191388348 | | Primary transcrpt | chr1: 191357505-191357706 | |
| hsa-mir-1279 | chr12: 57953203-67953265 | | | — | chr12: 67966611-67965812 | |
| hsa-mir-1294 | chr3: 71673812-716729230 | chr3: 71675507-716575076 | | Harpin | | |
| hsa-mir-1285-1 | chr7: 91671264-91671348 | chr7: 91713083-91712284 | | Primary transcrpt | | |
| hsa-mir-1290 | chr1: 11506121-19096229 | chr1: 19121702-19101903 | | Primary transcrpt | | |
| hsa-mir-1299 | chr9: 68290651-68292141 | | | — | chr9: 66291954-66292155 | |
| hsa-mir-1302-2 | chr15: 100318184-100316322 | | | — | chr15: 100319191-100319292 | |
| hsa-mir-1302-3 | chr2: 114037005-114057143 | | | — | chr2: 114056002-114058203 | |
| hsa-mir-1470 | chr19: 13421356-13421419 | | | — | | |
| hsa-mir-1826 | chr18: 23678008-33573093 | chr16: 33470673-33870774 | | Harpin | | |
| hsa-mir-1914 | chr20: 63042261-42043341 | | | — | chr20: 62043681-62043782 | |
| hsa-mir-1976 | chr1: 26723619-26752671 | | | — | chr1: 26733127-2653826 | |

| | Th1 GATA3 binding | | Th2 GATA3 binding | | |
|---|---|---|---|---|---|
| MicroRNA | Binding site 3 | Binding assignment | Binding site 1 | Binding site 2 | Binding assignment |
| hsa-mir-7-1 | | Primary transcrpt | | | — |
| hsa-mir-7-1 | | Harpin | chr1: 154656709-154656910 | | Harpin |
| hsa-mir-7-2 | | Harpin | | | — |
| hsa-mir-21 | | — | | | — |
| hsa-mir-23a | | Harpin | chr19: 13805746-13000947 | | Harpin |
| hsa-mir-24-2 | | Primary transcrpt | chr19: 13808748-13800947 | | Primary transcrpt |
| hsa-mir-25 | | Primary transcrpt | | | — |
| hsa-mir-26b | | Primary transcrpt | chr2: 18971032-218971233 | | Primary transcrpt |
| hsa-mir-26 | | Primary transcrpt and harpin | chr3: 189886932-189887133 | chr3: 189366652-189366853 | Primary transcrpt and harpin |
| hsa-mir-22 | | — | | | — |
| hsa-mir-23a | | Primary transcrpt | | | — |
| hsa-mir-92b | | — | | | — |
| hsa-mir-95 | | — | chr4: 6211604-8211807 | | Primary transcrpt |
| hsa-mir-138-2 | | Harpin | | | — |
| hsa-mir-142 | | Harpin | chr17: 53761283-53761484 | | Harpin |
| hsa-mir-142a | | Harpin | chr5: 169846219-169846420 | | Harpin |
| hsa-mir-152 | | Harpin | chr19: 64694125-54694326 | | Harpin |
| hsa-mir-151 | | Primary transcrpt | | | — |
| hsa-mir-155 | | Primary transcrpt and harpin | chr21: 25568031-25658232 | chr21: 25667950-25667751 | Primary transcrpt and harpin |
| hsa-mir-161a-1 | | Primary transcrpt | chr1: 197170662-197170963 | | Primary transcrpt |
| hsa-mir-166 | | — | | | — |
| hsa-mir-195 | | — | | | — |
| hsa-mir-200c | | Harpin | chr12: 6940496-6941097 | | Harpin |
| hsa-mir-205 | | — | | | — |
| hsa-mir-223 | | Harpin | | | — |
| hsa-mir-191 | | — | | | — |
| hsa-mir-120a | | — | | | — |
| hsa-mir-225 | | — | chrX: 76140950-76141151 | | Harpin |
| hsa-mir-330 | | — | | | — |
| hsa-mir-340 | | Primary transcrpt | chr5: 179431633-179431834 | | Primary transcrpt |
| hsa-mir-342 | | Primary transcrpt | chr14: 99602400-99602631 | | Primary transcrpt |
| hsa-mir-345 | | Harpin | | | — |
| hsa-mir-361 | | Primary transcrpt | | | — |
| hsa-mir-497 | | — | | | — |
| hsa-mir-548b | | — | | | — |
| hsa-mir-544c | | Primary transcrpt | chr12: 63289761-632899652 | | Primary transcrpt |
| hsa-mir-5450-1 | | Primary transcrpt | chr8: 124477843-124476022 | | Primary transcrpt |
| hsa-mir-546l | chr22: 25319429-25318230 | Primary transcrpt and harpin | | | — |
| hsa-mir-546n | | — | | | — |
| hsa-mir-546p | | — | | | — |
| hsa-mir-530-1 | | Primary transcrpt | | | — |
| hsa-mir-533 | | — | chr1: 100621719-100621920 | | Harpin |
| hsa-mir-565 | | — | | | — |
| hsa-mir-569 | | Primary transcrpt | | | — |
| hsa-mir-571 | | Primary transcrpt | | | — |
| hsa-mir-575 | | — | | | — |
| hsa-mir-581 | | Primary transcrpt | | | — |
| hsa-mir-583 | | — | | | — |
| hsa-mir-582 | | Harpin | chr6: 168626603-168626804 | | Harpin |
| hsa-mir-590 | | Primary transcrpt | | | — |
| hsa-mir-591 | | — | | | — |
| hsa-mir-596 | | Harpin | | | — |
| hsa-mir-595 | | — | | | — |
| hsa-mir-503 | | — | | | — |
| hsa-mir-513 | | Primary transcrpt | | | — |
| hsa-mir-517 | | Harpin | | | — |
| hsa-mir-620 | | Primary transcrpt | chr12: 115198390-115195591 | | Primary transcrpt |
| hsa-mir-621 | | — | chr13: 40259351-40259562 | | Primary transcrpt |
| hsa-mir-623 | | Harpin | chr13: 98804145-98804246 | chr12: 98806668-98606869 | Harpin |
| hsa-mir-626 | | — | | | — |
| hsa-mir-628 | | Primary transcrpt | | | — |
| hsa-mir-629 | | Primary transcrpt | | | — |
| hsa-mir-630 | | — | chr16: 70656213-70665414 | | Harpin |
| hsa-mir-633 | | Primary transcrpt | | | — |
| hsa-mir-634 | | Primary transcrpt | | | — |
| hsa-mir-639 | | — | chr19: 14501974-14502175 | | Primary transcrpt and harpin |
| hsa-mir-640 | | Primary transcrpt | | | — |
| hsa-mir-643 | | Primary transcrpt | chr19: 57464970-17484771 | | Primary transcrpt |
| hsa-mir-644 | | — | | | — |
| hsa-mir-647 | | Primary transcrpt | | | — |
| hsa-mir-648 | | — | chr22: 16643422-16643623 | | Harpin |
| hsa-mir-653 | | Harpin | chr22: 21493342-21493543 | | Harpin |
| hsa-mir-652 | | — | | | — |
| hsa-mir-663 | | Primary transcrpt and harpin | chr20: 26136944-26137445 | chr22: 26137970-26136171 | Primary transcrpt and harpin |
| hsa-mir-663b | | Harpin | chr21: 122723919-132729150 | | Harpin |
| hsa-mir-671 | | Primary transcrpt | chr7: 150561527-150561738 | | Primary transcrpt |
| hsa-mir-703 | | — | | | — |
| hsa-mir-765 | | Primary transcrpt and harpin | chrX: 115711043-115711244 | | Primary transcrpt |
| hsa-mir-874 | | — | | | — |
| hsa-mir-877 | | — | | | — |
| hsa-mir-865 | | — | chr3: 10412437-10412608 | | Harpin |
| hsa-mir-937 | | — | | | — |
| hsa-mir-938 | | — | | | — |
| hsa-mir-942 | | Primary transcrpt | | | — |
| hsa-mir-943 | | Primary transcrpt | | | — |
| hsa-mir-1181 | | Primary transcrpt and harpin | | | — |
| hsa-mir-1200 | | Primary transcrpt | chr7: 374537539-37453960 | | Primary transcrpt |
| hsa-mir-1206 | | Harpin | chr1: 129040455-129040656 | chr6: 129041910-129042111 | Harpin |
| hsa-mir-1233 | | Primary transcrpt | chr16: 22515870-32516071 | | Primary transcrpt |
| hsa-mir-1244 | | Primary transcrpt | chr2: 232281107-232281205 | | Primary transcrpt |
| hsa-mir-1248 | | — | | | — |
| hsa-mir-1249 | | Primary transcrpt | | | — |
| hsa-mir-1255a | | Primary transcrpt and harpin | chr4: 102455540-102455741 | | Primary transcrpt |
| hsa-mir-1256 | | Primary transcrpt | | | — |
| hsa-mir-1270 | | Primary transcrpt | chr19: 20299576-20299777 | | Primary transcrpt |
| hsa-mir-1277 | | — | chrX: 117364900-117366001 | chrX: 1173646257-117366458 | Primary transcrpt |
| hsa-mir-1278 | | Primary transcrpt | | | — |
| hsa-mir-1279 | | Harpin | | | — |
| hsa-mir-1294 | | — | | | — |
| hsa-mir-1285-1 | | — | | | — |
| hsa-mir-1290 | | — | | | — |
| hsa-mir-1299 | | Harpin | | | — |
| hsa-mir-1302-2 | | Harpin | chr15: 100319191-100319892 | | Harpin |
| hsa-mir-1302-3 | | Harpin | chr2: 114058017-114088218 | | Harpin |
| hsa-mir-1470 | | — | chr19: 15423447-15423648 | | Harpin |
| hsa-mir-1826 | | — | | | — |
| hsa-mir-1914 | | Harpin | | | — |
| hsa-mir-1976 | | Harpin | | | — |

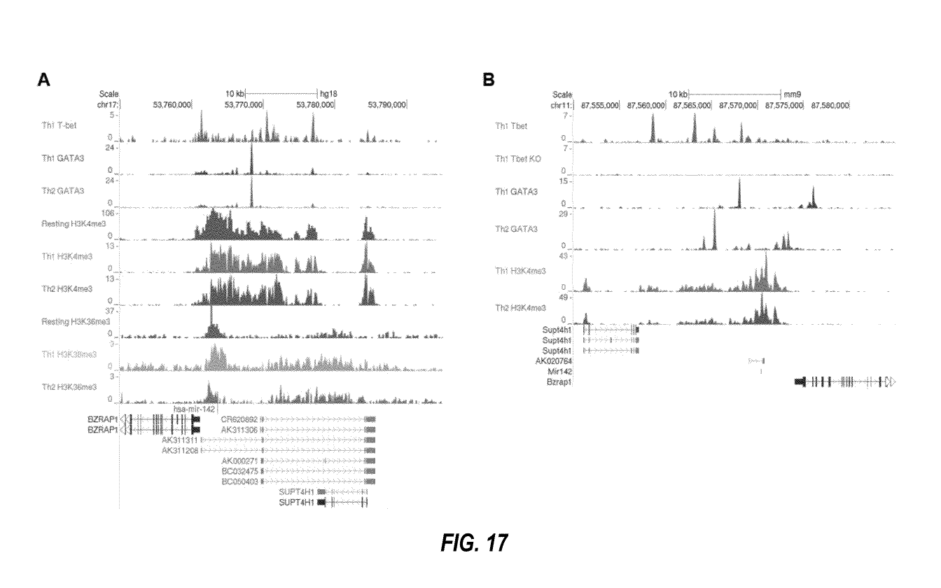

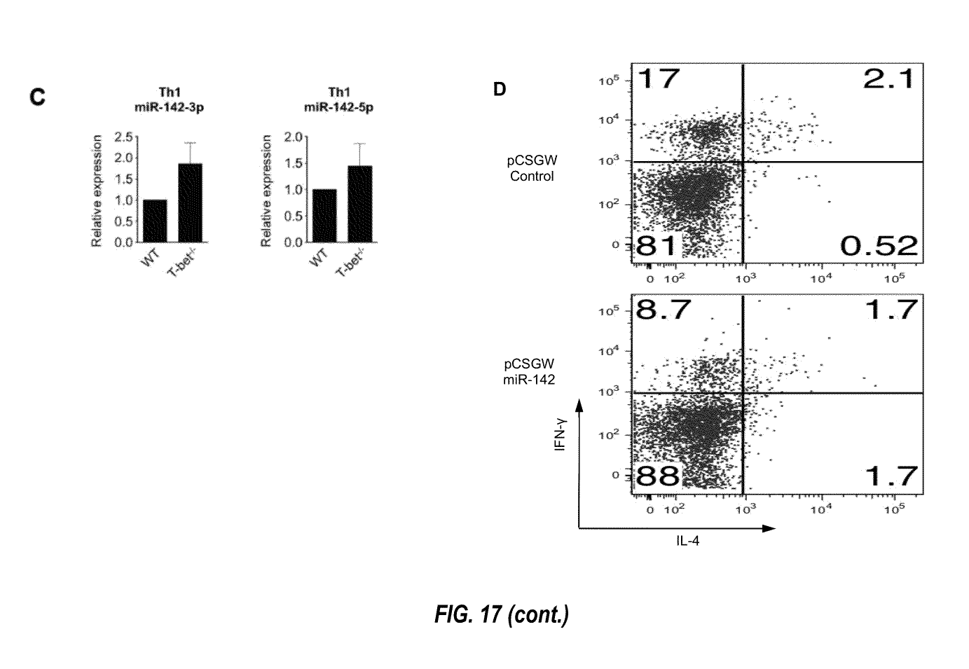

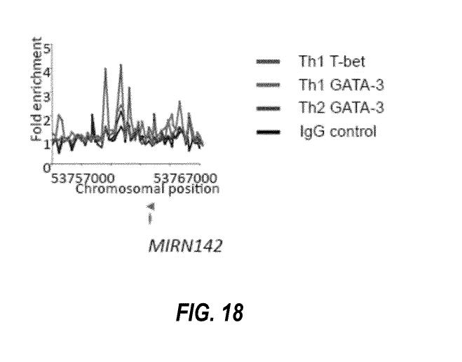

Our ChIP-seq data demonstrated binding of both T-bet and GATA-3 at the mir-142 locus (FIG. 17a). Binding of T-bet and GATA-3 at this region was also observed upon re-analysis of our previously published ChIP-chip datasets (Jenner et al., 2009) (FIG. 18). mir-142 is a highly-conserved miRNA that exhibits an expression pattern restricted to haematopoietic lineages (Landgraf et al., 2007). Analysis of histone modifications in these cell subsets demonstrated enrichment of methylation of histone H3 at lysine 4 (H3K4me3), a transcriptional initiation marker, across the mir-142 locus in both resting and Th1 and Th2 human T cells. Methylation of histone H3 at lysine 36 (H3K36me3) in these cell subsets demonstrated that there is active transcriptional elongation across the mir-142 locus. To determine if the regulation of mir-142 expression was conserved across species, we examined transcription factor occupancy and histone marks in murine CD4⁺ T cells. This analysis revealed a similar pattern of transcription factor binding and active transcription at the murine mir-142 locus (FIG. 17b). These data demonstrated that mir-142 is a target of the regulatory transcription factors that control T cell lineage commitment and that active transcription occurs in naïve and effector lineages in both human and mouse. We therefore screened for functional effects of mir-142 as a potential component of these pathways. Mir-142 expression was increased in T-bet^{-/-} Th1 cells (FIG. 17c), suggesting that T-bet was able to repress the expression of this microRNA. Mir-142 expression was unchanged in Th1 and Th2 cells from OX40-Cre x GATA-3^{fl/fl} mice, supporting a role in Th1 rather than Th2 biology (data not shown). Lentivirus-mediated expression of mir-142 in human CD4⁺ T cells under Th1-polarizing conditions specifically inhibited IFN-γ production (FIG. 17d), suggesting an important role of this molecule in controlling cytokine expression.

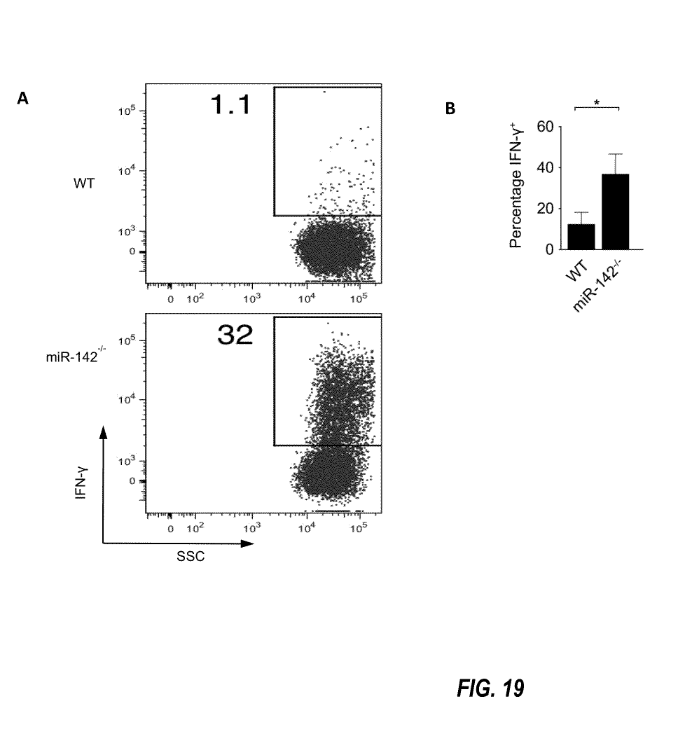

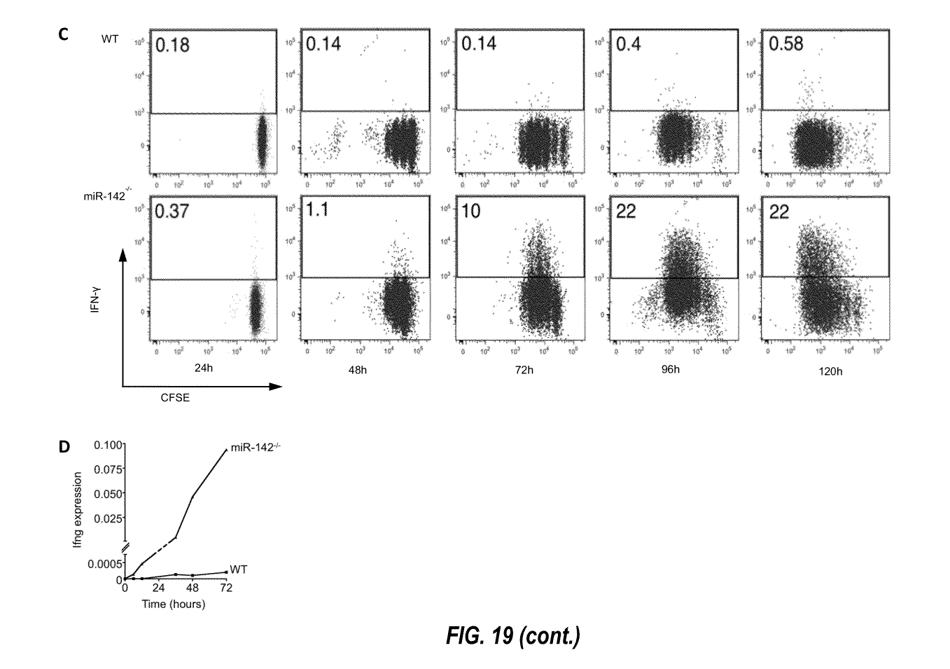

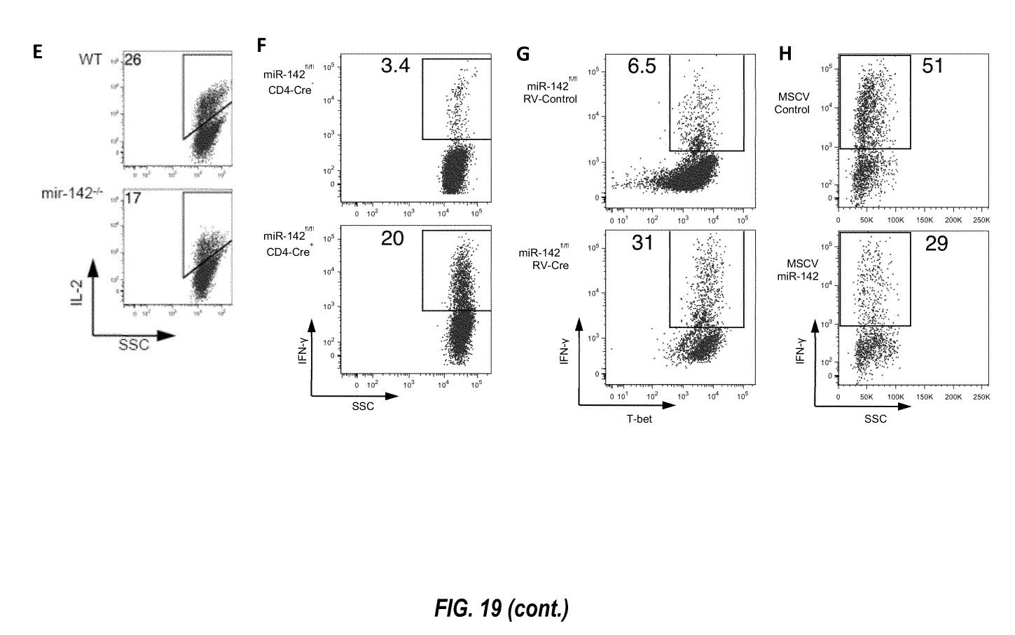

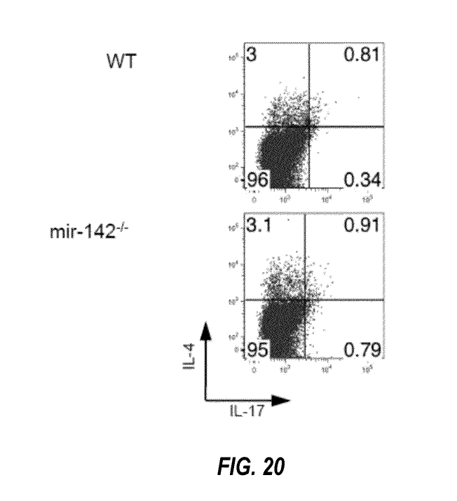

These data show that mir-142 is capable of regulating the process of normal T helper cell differentiation. Therefore, we further examined the role of mir-142 in T cell development using both constitutive mir-142 deficient mice (mir-142^{-/-}) and conditional mir-142 deficient mice (mir-142^{fl/fl}) in which the mir-142 locus is flanked by LoxP sites. In addition to the phenotype described here, mir-142^{-/-} mice display a specific defect in unactivated naïve T cell homeostasis in vivo (manuscript under revision). Upon activation of mir-142^{-/-} Thp under non-polarising Th0 conditions, significant hyperproduction of IFN-γ was seen when compared with WT (FIG. 19a-b). This was not accompanied by upregulation of IL-4 or IL-17, indicating default acquisition of a Th1 phenotype (FIG. 20). IFN-γ mRNA and protein were not expressed by either WT or mir-142^{-/-} Thp directly ex vivo and CFSE dilution demonstrated normal proliferation but markedly increased IFN-γ production at all divisions in vitro (FIG. 19c-d). IL-2 production was reduced in mir-142^{-/-} T cells (FIG. 19e) although the survival and rates of apoptosis of mir-142^{-/-} T cells were unchanged compared to WT, even at suboptimal levels of T cell receptor and CD28 ligation (not shown). Thp purified from CD4-Cre x mir-142^{fl/fl} showed an identical increase in expression of IFN-γ, demonstrating a T cell-intrinsic requirement for mir-142 (FIG. 19f). To determine whether mir-142 was required for normal T cell function following activation, we deleted mir-142 after in vitro stimulation of mir-142^{fl/fl} Thp using retroviral expression of Cre recombinase, which resulted in elevated production of IFN-γ (FIG. 19g). To confirm that this lineage commitment abnormality was caused directly by mir-142 deficiency, we recomplemented mir-142^{-/-} Thp by retroviral transduction of mir-142, which resulted in a marked reduction in the expression of IFN-γ (FIG. 19h).

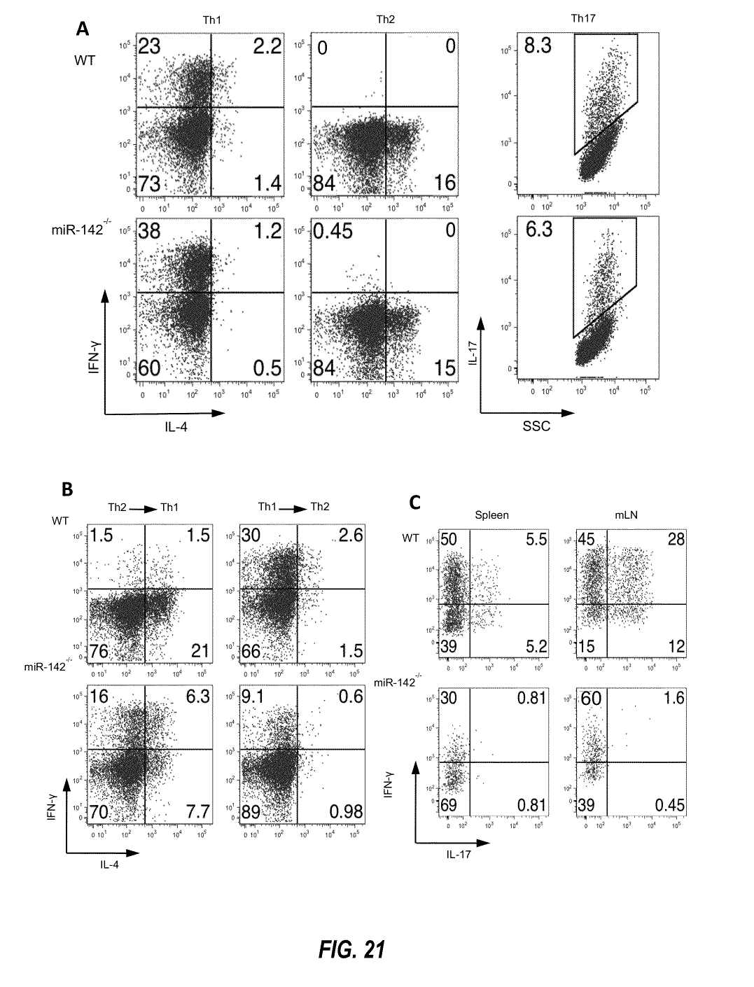

Given this abnormal default lineage commitment under non-polarising conditions, we performed in vitro skewing assays and found that Th1, Th2 and Th17 cells could all be generated from mir-142^{-/-} Thp, although IFN-γ production was higher in mir-142^{-/-} cells under Th1 conditions (FIG. 21a). Expression of the lineage-defining cytokines IL4 and IL17 was comparable in both genotypes, indicating a specific role for mir-142 in controlling Th1 differentiation. There is some evidence to suggest that microRNAs may have a role in cellular plasticity and stability after an initial lineage choice has been made (Takahashi et al., 2012). In order to address this hypothesis, we performed in vitro crossover experiments in which cells were initially cultured in either Th1 or Th2 skewing conditions and then switched at day 3 to the opposing subtype conditions. Wild type cells were completely stable when sequentially cultured in the opposite skewing conditions. In contrast, mir-142^{-/-} T cells were far more unstable, being unable to maintain their initial lineage choice (FIG. 21b). This demonstrates that mir-142 expression is necessary for the maintenance of lineage stability. To examine the effect of mir-142 on T cell lineage differentiation in vivo, we adoptively transferred WT and mir-142^{-/-} Thp into RAG-1-deficient mice in which the cells undergo homeostatic expansion. We found that WT cells produced both IL-17 and IFN-γ at 4 weeks post-transfer whilst mir-142^{-/-} cells produced only IFN-γ but no IL-17 (FIG. 21 c). These data demonstrate that the in vitro findings of default Th1 differentiation in the absence of mir-142 also occur in vivo.

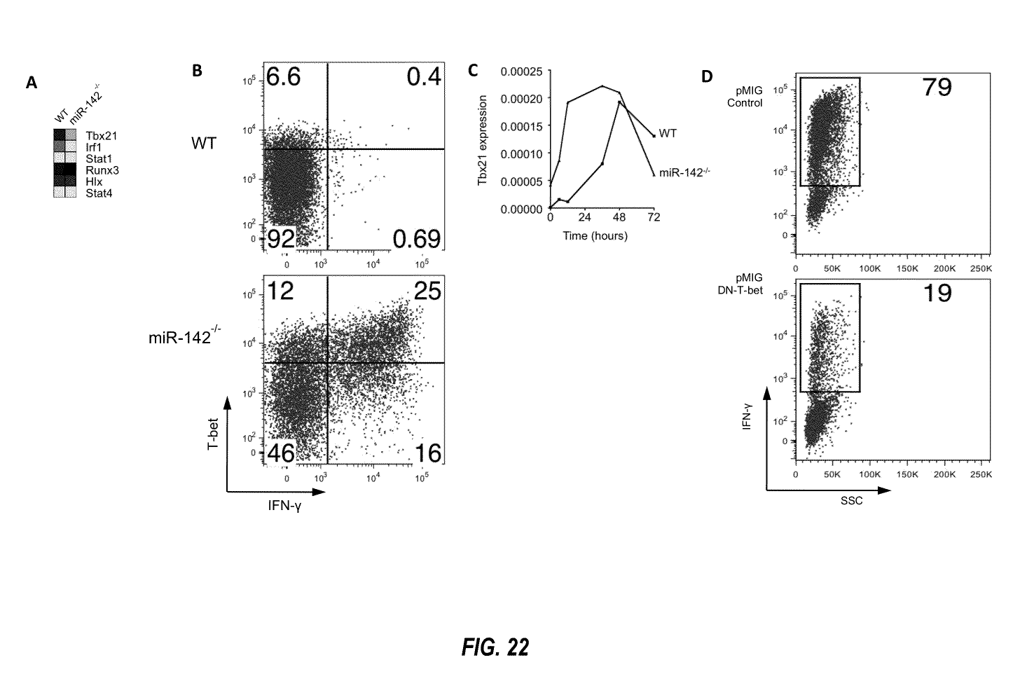

To identify target genes responsible for aberrant lineage commitment, we performed microarray profiling of WT and mir-142^{-/-} Thp that had been activated in vitro for 36 hours. Among genes known to be involved in Th1 development, we found substantially increased Tbx21 transcript levels in the absence of mir-142 (FIG. 22a). In addition, protein levels of Tbx21 were also markedly elevated in the absence of mir-142 (FIG. 22b). A time course analysis by quantitative PCR demonstrated an early, accelerated rise in T-bet expression in mir-142^{-/-} T cells compared with WT (FIG. 22c), before IFN-γ was detected. This finding rules out that the augmented induction of T-bet expression at an early time point is due to IFN-γ signalling. Mir-142 mediated suppression of a luciferase reporter gene via the T-bet 3′UTR containing these multiple sites (FIG. 22c). We sought to determine whether the mechanism of default Th1 lineage commitment in mir-142^{-/-} T cells was dependent on T-bet. We used a dominant negative approach to suppress T-bet function, as described previously (Mullen et al., 2002). Expression of dominant negative (DN)-T-bet in mir-142^{-/-} T cells prevented default expression of IFN-γ, demonstrating that a regulatory axis incorporating both T-bet and mir-142 is critical for the development of this abnormal phenotype (FIG. 22d).

In summary, these data identify a previously undescribed interaction between transcription factors and microRNAs required for T cell lineage commitment. Mir-142 is critically important for both T cell differentiation and the stability of Th1 cells. In situations where the plasticity and lineage commitment of T cells is of pathological significance, modulation of mir-142 activity would be expected to produce therapeutic benefit.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that changes can be made without departing from the spirit and scope of the invention(s).

### 6. REFERENCES

1. Takada, K. & Jameson, S. C. Naive T cell homeostasis: from awareness of space to a sense of place. Nat Rev Immunol 9, 823-832 (2009).
2. Jameson, S. C. Maintaining the norm: T-cell homeostasis. Nat Rev Immunol 2, 547-556 (2002).
3. Chen, C.-Z., Li, L., Lodish, H. F. & Bartel, D. P. MicroRNAs modulate hematopoietic lineage differentiation. Science 303, 83-86 (2004).
4. Cobb, B. S. et al. A role for Dicer in immune regulation. J Exp Med 203, 2519-2527 (2006).
5. Muljo, S. A. et al. Aberrant T cell differentiation in the absence of Dicer. J Exp Med 202, 261-269 (2005).
6. Landgraf, P. et al. A mammalian microRNA expression atlas based on small RNA library sequencing. Cell 129, 1401-1414 (2007).
7. Cho, B. K., Rao, V. P., Ge, Q., Eisen, H. N. & Chen, J. Homeostasis-stimulated proliferation drives naive T cells to differentiate directly into memory T cells. J Exp Med 192, 549-556 (2000).
8. Purton, J. F. et al. Antiviral CD4+ memory T cells are IL-15 dependent. J Exp Med 204, 951-961 (2007).
9. Tomita, T. et al. IL-7 is essential for lymphopenia-driven turnover of colitogenic CD4(+) memory T cells in chronic colitis. Eur J Immunol 39, 2737-2747 (2009).
10. Powrie, F. et al. Inhibition of Th1 responses prevents inflammatory bowel disease in scid mice reconstituted with CD45RBhi CD4+ T cells. Immunity 1, 553-562 (1994).
11. Guimond, M. et al. Interleukin 7 signaling in dendritic cells regulates the homeostatic proliferation and niche size of CD4+ T cells. Nat Immunol 10, 149-157 (2009).
12. Osborne, L. C., Patton, D. T., Seo, J. H. & Abraham, N. Elevated IL-7 availability does not account for T cell proliferation in moderate lymphopenia. The Journal of Immunology 186, 1981-1988 (2011).
13. Palmer, M. J. et al. Interleukin-7 receptor signaling network: an integrated systems perspective. Cell. Mol. Immunol. 5, 79-89 (2008).
14. Onishi, M. et al. Identification and characterization of a constitutively active STAT5 mutant that promotes cell proliferation. Mol. Cell. Biol. 18, 3871-3879 (1998).
15. Tamiya, T., Kashiwagi, I., Takahashi, R., Yasukawa, H. & Yoshimura, A. Suppressors of cytokine signaling (SOCS) proteins and JAK/STAT pathways: regulation of T-cell inflammation by SOCS1 and SOCS3. Arterioscler. Thromb. Vasc. Biol. 31, 980-985 (2011).
16. Fletcher, T. C., DiGiandomenico, A. & Hawiger, J. Extended anti-inflammatory action of a degradation-resistant mutant of cell-penetrating suppressor of cytokine signaling 3. Journal of Biological Chemistry 285, 18727-18736 (2010).
17. Enright, A. J. et al. MicroRNA targets in Drosophila. Genome Biol 5, R1 (2003).
18. Betel, D., Wilson, M., Gabow, A., Marks, D. S. & Sander, C. The microRNA.org resource: targets and expression. Nucleic Acids Res. 36, D149-53 (2008).
19. Vella, A., Teague, T. K., Ihle, J., Kappler, J. & Marrack, P. Interleukin 4 (IL-4) or IL-7 prevents the death of resting T cells: stat6 is probably not required for the effect of IL-4. J Exp Med 186, 325-330 (1997).
20. Carrette, F. & Surh, C. D. IL-7 signaling and CD127 receptor regulation in the control of T cell homeostasis. Semin. Immunol. 24, 209-217 (2012).
21. Park, J.-H. et al. Suppression of IL7Ralpha transcription by IL-7 and other prosurvival cytokines: a novel mechanism for maximizing IL-7-dependent T cell survival. Immunity 21, 289-302 (2004).
22. Voehringer, D., Liang, H.-E. & Locksley, R. M. Homeostasis and effector function of lymphopenia-induced ‘memory-like’ T cells in constitutively T cell-depleted mice. J Immunol 180, 4742-4753 (2008).
23. Endo, T. A. et al. A new protein containing an SH2 domain that inhibits JAK kinases. Nature 387, 921-924 (1997).
24. Hilton, D. J. et al. Twenty proteins containing a C-terminal SOCS box form five structural classes. Proc Natl Acad Sci USA 95, 114-119 (1998).
25. Zhang, J. G. et al. The conserved SOCS box motif in suppressors of cytokine signaling binds to elongins B and C and may couple bound proteins to proteasomal degradation. Proc Natl Acad Sci USA 96, 2071-2076 (1999).
26. Pellegrini, M. et al. IL-7 engages multiple mechanisms to overcome chronic viral infection and limit organ pathology. Cell 144, 601-613 (2011).
27. Matsumoto, A. et al. A role of suppressor of cytokine signaling 3 (SOCS3/CIS3/SSI3) in CD28-mediated interleukin 2 production. J Exp Med 197, 425-436 (2003).
28. Kedde, M. et al. RNA-binding protein Dnd1 inhibits microRNA access to target mRNA. Cell 131, 1273-1286 (2007).
29. Arvey, A., Larsson, E., Sander, C., Leslie, C. S. & Marks, D. S. Target mRNA abundance dilutes microRNA and siRNA activity. Mol. Syst. Biol. 6, 363 (2010).
30. Watanabe, M. et al. Interleukin 7 is produced by human intestinal epithelial cells and regulates the proliferation of intestinal mucosal lymphocytes. Journal of Clinical Investigation 95, 2945-2953 (1995).
31. Gregory, S. G. et al. Interleukin 7 receptor alpha chain (IL7R) shows allelic and functional association with multiple sclerosis. Nat Genet. 39, 1083-1091 (2007).
32. Lundmark, F. et al. Variation in interleukin 7 receptor alpha chain (IL7R) influences risk of multiple sclerosis. Nat Genet. 39, 1108-1113 (2007).
33. Hafler, D. A. et al. Risk alleles for multiple sclerosis identified by a genomewide study. N Engl J Med 357, 851-862 (2007).
34. Anderson, C. A. et al. Meta-analysis identifies 29 additional ulcerative colitis risk loci, increasing the number of confirmed associations to 47. Nat Genet. 43, 246-252 (2011).
35. Crawley, A. M. & Angel, J. B. Expression of γ-chain cytokine receptors on CD8(+) T cells in HIV infection with a focus on IL-7Rα (CD127). Immunology and Cell Biology (2011).doi:10.1038/icb.2011.66
36. Mackall, C. L., Fry, T. J. & Gress, R. E. Harnessing the biology of IL-7 for therapeutic application. Nat Rev Immunol 11, 330-342 (2011).
37. Irizarry, R. A. et al. Exploration, normalization, and summaries of high density oligonucleotide array probe level data. Biostatistics 4, 249-264 (2003).
38. Huber, W., Heydebreck, von, A., Sültmann, H., Poustka, A. & Vingron, M. Variance stabilization applied to microarray data calibration and to the quantification of differential expression. Bioinformatics 18 Suppl 1, S96-104 (2002).
39. Battke, F., Symons, S. & Nieselt, K. Mayday—integrative analytics for expression data. BMC Bioinformatics 11, 121 (2010).
Cordes, K. R., N. T. Sheehy, M. P. White, E. C. Berry, S. U. Morton, A. N. Muth, T.-H. Lee, J. M. Miano, K. N. Ivey, and D. Srivastava. 2009. miR-145 and miR-143 regulate smooth muscle cell fate and plasticity. Nature. 460:705-710.
Ding, Y., and C. E. Lawrence. 2003. A statistical sampling algorithm for RNA secondary structure prediction. Nucleic Acids Res. 31:7280-7301.
Ding, Y., C. Y. Chan, and C. E. Lawrence. 2005. RNA secondary structure prediction by centroids in a Boltzmann weighted ensemble. RNA. 11:1157-1166.
Fontenot, J. D., M. A. Gavin, and A. Y. Rudensky. 2003. Foxp3 programs the development and function of CD4+CD25+ regulatory T cells. Nat. Immunol. 4:330-336.
Hori, S., T. Nomura, and S. Sakaguchi. 2003. Control of regulatory T cell development by the transcription factor Foxp3. Science. 299:1057-1061.
Irizarry, R. A., B. Hobbs, F. Collin, Y. D. Beazer-Barclay, K. J. Antonellis, U. Scherf, and T. P. Speed. 2003. Exploration, normalization, and summaries of high density oligonucleotide array probe level data. Biostatistics. 4:249-264.
Ivanov, L I., B. S. Mckenzie, L. Zhou, C. E. Tadokoro, A. Lepelley, J. J. Lafaille, D. J. Cua, and D. R. Littman. 2006. The orphan nuclear receptor RORgammat directs the differentiation program of proinflammatory IL-17+ T helper cells. Cell. 126:1121-1133.
Jenner, R. G., M. J. Townsend, I. Jackson, K. Sun, R. D. Bouwman, R. A. Young, L. H. Glimcher, and G. M. Lord. 2009. The transcription factors T-bet and GATA-3 control alternative pathways of T-cell differentiation through a shared set of target genes. Proc Natl Acad Sci USA. 106:17876-17881.
Kertesz, M., N. Iovino, U. Unnerstall, U. Gaul, and E. Segal. 2007. The role of site accessibility in microRNA target recognition. Nat. Genet. 39:1278-1284.
Khattri, R., T. Cox, S.-A. Yasayko, and F. Ramsdell. 2003. An essential role for Scurfin in CD4+CD25+ T regulatory cells. Nat. Immunol. 4:337-342.
Landgraf, P., M. Rusu, R. Sheridan, A. Sewer, N. Iovino, A. Aravin, S. Pfeffer, A. Rice, A. O. Kamphorst, M. Landthaler, C. Lin, N. D. Socci, L. Hermida, V. Fulci, S. Chiaretti, R. Foà, J. Schliwka, U. Fuchs, A. Novosel, R.-U. Müller, B. Schermer, U. Bissels, J. Inman, Q. Phan, M. Chien, D. B. Weir, R. Choksi, G. de Vita, D. Frezzetti, H.-I. Trompeter, V. Hornung, G. Teng, G. Hartmann, M. Palkovits, R. Di Lauro, P. Wernet, G. Macino, C. E. Rogler, J. W. Nagle, J. Ju, F. N. Papavasiliou, T. Benzing, P. Lichter, W. Tam, M. J. Brownstein, A. Bosio, A. Borkhardt, J. J. Russo, C. Sander, M. Zavolan, and T. Tuschl. 2007. A mammalian microRNA expression atlas based on small RNA library sequencing. Cell. 129:1401-1414.
Muljo, S. A., K. M. Ansel, C. Kanellopoulou, D. M. Livingston, A. Rao, and K. Rajewsky. 2005. Aberrant T cell differentiation in the absence of Dicer. J Exp Med. 202:261-269.
Mullen, A. C., A. S. Hutchins, F. A. High, H. W. Lee, K. J. Sykes, L. A. Chodosh, and S. L. Reiner. 2002. Hlx is induced by and genetically interacts with T-bet to promote heritable TH1 gene induction. Nat. Immunol. 3:652-658.
O'Shea, J. J., and W. E. Paul. 2010. Mechanisms underlying lineage commitment and plasticity of helper CD4+ T cells. Science. 327:1098-1102.
Rehmsmeier, M., P. Steffen, M. Hochsmann, and R. Giegerich. 2004. Fast and effective prediction of microRNA/target duplexes. RNA. 10:1507-1517.
Szabo, S. J., S. T. Kim, G L Costa, X. Zhang, C. G. Fathman, and L. H. Glimcher. 2000. A novel transcription factor, T-bet, directs Th1 lineage commitment. Cell. 100:655-669.
Takahashi, H., T. Kanno, S. Nakayamada, K. Hirahara, G. Sciumè, S. A. Muljo, S. Kuchen, R. Casellas, L. Wei, Y. Kanno, and J. J. O'Shea. 2012. TGF-β and retinoic acid induce the microRNA miR-10a, which targets Bcl-6 and constrains the plasticity of helper T cells. Nat. Immunol. 13:587-595.
Zhang, D. H., L. Cohn, P. Ray, K. Bottomly, and A. Ray. 1997. Transcription factor GATA-3 is differentially expressed in murine Th1 and Th2 cells and controls Th2-specific expression of the interleukin-5 gene. J Biol Chem. 272:21597-21603.
Zhang, Y., T. Liu, C. A. Meyer, J. Eeckhoute, D. S. Johnson, B. E. Bernstein, C. Nusbaum, R. M. Myers, M. Brown, W. Li, and X. S. Liu. 2008. Model-based analysis of ChIP-Seq (MACS). Genome Biol. 9:R137.
Zheng, W., and R. A. Flavell. 1997. The transcription factor GATA-3 is necessary and sufficient for Th2 cytokine gene expression in CD4 T cells. Cell. 89:587-596.
Zhou, L., M. M. W. Chong, and D. R. Littman. 2009. Plasticity of CD4+ T cell lineage differentiation. Immunity. 30:646-655.

1. A method of treating an autoimmune disease in a subject, comprising administering to the subject a compound comprising an oligonucleotide, wherein the oligonucleotide comprises a first strand that consists of 8 to 200 nucleosides, and wherein the first strand comprises a region that is identical to at least 8 contiguous nucleotides of miR-142.

2. The method of claim 1, wherein the autoimmune disease is selected from rheumatoid arthritis, multiple sclerosis, psoriasis, and inflammatory bowel disease.

3. A method of attenuating rejection of a transplanted organ in a subject, comprising administering to the subject a compound comprising an oligonucleotide, wherein the oligonucleotide comprises a first strand that consists of 8 to 200 nucleosides, and wherein the first strand comprises a region that is identical to at least 8 contiguous nucleotides of miR-142.

4. (canceled)

5. A method of increasing regulatory T cell production and/or IgM production and/or enhancing IL-7 receptor signaling in a subject, comprising administering to the subject a compound comprising an oligonucleotide, wherein the oligonucleotide comprises a first strand that consists of 8 to 200 nucleosides, and wherein the first strand comprises a region that is identical to at least 8 contiguous nucleotides of miR-142.

6. The method of claim 5, wherein the subject is a subject with HIV.

7. (canceled)

8. (canceled)

9. The method of claim 1, wherein the miR-142 is miR-142-5p or miR-142-3p.

10. (canceled)

11. (canceled)

12. The method of claim 9, wherein the first strand comprises a region that is identical to a seed match region of miR-142-3p or miR-142-5p.

13. (canceled)

14. The method of claim 1, wherein the oligonucleotide further comprises a second strand that is complementary to at least a portion of the first strand.

15. The method of claim 14, wherein the second strand comprises at least one modified nucleoside.

16. The method of claim 1, comprising administering to the subject a vector that encodes an shRNA, wherein the shRNA comprises a region that is identical to at least 8 contiguous nucleotides of miR-142.

17.-22. (canceled)

23. A method of enhancing immune response in a subject, comprising administering to the subject a compound comprising an oligonucleotide, wherein the oligonucleotide consists of 8 to 200 nucleosides, and wherein the oligonucleotide comprises a region that is complementary to at least 8 contiguous nucleotides of miR-142.

24. The method of claim 23, wherein the subject has cancer.

25. The method of claim 24, wherein the cancer is selected from hematologic malignancies and dysplasias such as acute and chronic myeloid leukemia, acute and chronic lymphocytic leukemia, myelodysplasia, Hodgkin's and non-Hodgkin's lymphoma, multiple myeloma and Waldenstrom's macroglobulinemia, myeloproliferative disorders such as myelofibrosis and polycythemia rubra vera; solid tumors such as small-cell and non-small cell lung cancer, breast cancer, colorectal cancer, prostate cancer, ovarian cancer, gastric and esophageal cancer, glioblastoma multiforme, head and neck cancer, pancreatic cancer, hepatocellular carcinoma, soft tissue sarcoma, melanoma, bladder cancer, and renal cancer.

26. The method of claim 23, wherein the subject has an infection.

27. The method of claim 26, wherein the infection is an intracellular infection.

28. The method of claim 26, wherein the infection is a viral infection, bacterial infection, or parasitic infection.

29. The method of claim 23, wherein the subject has received a vaccine before or at the same time as administration of the compound.

30. A method of reducing regulatory T cell production and/or reducing IgM antibody production and/or inhibiting IL-7 receptor signaling in a subject, comprising administering to the subject a compound comprising an oligonucleotide, wherein the oligonucleotide consists of 8 to 200 nucleosides, and wherein the oligonucleotide comprises a region that is complementary to at least 8 contiguous nucleotides of miR-142.

31. The method of claim 30, wherein the subject has acute lymphoblastic leukemia (ALL). contiguous nucleotides of miR 112.

32. (canceled)

33. (canceled)

34. The method of claim 23, wherein miR-142 is miR-142-5p or miR-142-3p.

35.-37. (canceled)

38. The method of claim 34, wherein the oligonucleotide comprises a region that is complementary to a seed match region of miR-142-3p or miR-142-5p.

39. (canceled)

40. (canceled)

41. The method of claim 23, wherein the oligonucleotide comprises at least one modified nucleoside.

42.-51. (canceled)


SEQUENCE LISTING (3 sequences)

SEQ ID NO: 1 - RNA, 21 nt, Artificial sequence
  Synthetic
cauaaaguagaaagcacuacu

SEQ ID NO: 2 - RNA, 23 nt, Artificial sequence
  Synthetic
uguaguguuuccuacuuuaugga

SEQ ID NO: 3 - RNA, 87 nt, Artificial sequence
  Synthetic
gacagugcagucacccauaaaguagaaagcacuacuaacagcacuggaggguguaguguuuccuacuuuauggaugaguguacugug